(19) United States
(12) Patent Application Publication
(10) Pub. No.: US 2026/0245674 A1
(43) Pub. Date: Aug. 20, 2026

(54) ENSEMBLE-FREE, UNCERTAINTY-DRIVEN ACTIVE LEARNING FOR GENERATING CONCISE YET COMPREHENSIVE DATA SETS FOR TRAINING MACHINE LEARNED INTERATOMIC POTENTIALS

(71) Applicant: NEC Laboratories Europe GmbH, Heidelberg (DE)

(72) Inventors: Viktor ZAVERKIN, Heidelberg (DE); Henrik CHRISTIANSEN, Heidelberg (DE); Federico ERRICA, Heidelberg (DE); Francesco ALESIANI, Heidelberg (DE)

(21) Appl. No.: 19/161,322

(22) PCT Filed: Feb. 15, 2024
(86) PCT No.: PCT/IB2024/051431

§ 371 Date: Sep. 2, 2025

Related U.S. Application Data
(60) Provisional application No. 63/541,313, filed on Sep. 29, 2023.

(51) Int. Cl. G16C 20/70; G16C 10/00

(52) U.S. Cl. CPC G16C 20/70; G16C 10/00

(57) ABSTRACT

A computer-implemented, machine learning (ML) method for training interatomic potentials of an atomic system includes performing uncertainty-driven active learning. The uncertainty-driven active learning includes running an atomistic simulation using ensemble-free uncertainties to generate unlabeled atomic data. The ensemble-free uncertainties are derived from gradient features and used to terminate the atomistic simulation and bias the atomistic simulation toward unexplored regions of a configurational and/or chemical space of the atomic system. The uncertainty-driven active learning further includes performing active learning to select an atomic configuration from the unlabeled atomic data using a trajectory of the atomistic simulation and to train an ML model for the interatomic potentials using the atomic configuration. The method can be used in a variety of applications in artificial intelligence (AI) including, but not limited to, several anticipated use cases in medical diagnostics/applications and in healthcare, for example, to optimize predictions or support decision-making.

### CROSS-REFERENCE TO RELATED APPLICATIONS

This application is a U.S. National Phase application under 35 U.S.C. § 371 of International Application No. PCT/IB2024/051431, filed on Feb. 15, 2024, and claims benefit to U.S. Provisional Application No. 63/541,313, filed on Sep. 29, 2023, the entire contents of which is hereby incorporated by reference herein. The International Application was published in English on Apr. 3, 2025 as WO 2025/068773 A1 under PCT Article 21(2).

### FIELD

The present invention relates to Artificial Intelligence (AI) and machine learning (ML), and in particular to a method, system, data structure, computer program product and computer-readable medium for generating data sets for training machine learned interatomic potentials, with applications to discovery of new generation energy, polymer materials and drug design. In general, the present invention can be used in a variety of applications including, but not limited to, several anticipated use cases in medical diagnostics/applications and in healthcare for example, to optimize predictions or support decision-making.

### BACKGROUND

Machine learning has transformed the way interatomic interactions are modeled in complex molecular and bulk material systems. The effectiveness of machine learning technology relies on providing well-parameterized interatomic potential models trained on high-quality first-principles data, i.e., energies and atomic forces computed with density-functional-theory-based (DFT) methods. The generation of a concise yet comprehensive training data set is a technical challenge because of the high computational cost of DFT (small systems sizes) and limited computational efficiency (long simulation times) of conventional sampling algorithms such as molecular dynamics (MD) or Monte Carlo (MC) simulations. Particularly, a single DFT calculation of energies and atomic forces may require computation times ranging from seconds to hours, depending on the size of the investigated atomic systems, which can vary from less than fifty atoms for small drugs to several thousand atoms for biological matter. Exploring the relevant phase space with MD may require running simulations for several millions of time steps, making the combination with DFT often not tractable.

Combining machine-learned interatomic potentials (MLIPs) with active learning (AL) may accelerate the generation of a training data set for training them (see Zhang, L., et al., “Active learning of uniformly accurate interatomic potentials for materials simulation,” Phys. Rev. Mater. 3, 023804 (2019); and Vandermause, J., et al., “On-the-fly active learning of interpretable Bayesian force fields for atomistic rare events,” npj Comput. Mater. 6 (2020), each of which is hereby incorporated by reference herein). This involves running an MD simulation using energies and atomic forces from an MLIP until a threshold value, typically a high uncertainty of the employed MLIP, is reached. For an atomic configuration with highest uncertainty, energy and atomic forces are evaluated using a pre-defined DFT method. In this setting, it is crucial to know physical conditions, such as temperature and pressure, at which all relevant configurational changes will occur. Otherwise, the number of MD time steps can be a limiting factor for generating complete training data sets. Employing enhanced sampling techniques (see Vandenhaute, S., et al., “Machine learning potentials for metal-organic frameworks using an incremental learning approach,” npj Comput. Mater. 9, 19 (2023), which is hereby incorporated by reference herein), such as metadynamics, to accelerate exploration of all relevant conformational changes is limited, as it again requires knowing these conformational changes beforehand to define so-called collective variables (CVs). Besides the limitations introduced by MD simulations, using an ensemble of models is a typical computational bottleneck of approaches according to existing technology for generating training data sets. This also holds for approaches utilizing uncertainty-biases to explore conformational space (see Schran, C., et al., “Committee neural network potentials control generalization errors and enable active learning,” J. Chem. Phys. 153, 104105 (2020); Kulichenko, M., et al., “Uncertainty-driven dynamics for active learning of interatomic potentials,” Nat. Comput. Sci. 3, 230-239 (2023); and Van der Oord, C., et al., “Hyperactive Learning (HAL) for Data-Driven Interatomic Potentials,” ArXiv abs/2210.04225 (2022), each of which is hereby incorporated by reference herein).

### SUMMARY

In an embodiment, the present invention provides a computer-implemented, machine learning (ML) method for training interatomic potentials of an atomic system that includes performing uncertainty-driven active learning. The uncertainty-driven active learning includes running an atomistic simulation of the atomic system using ensemble-free uncertainties to generate unlabeled atomic data. The ensemble-free uncertainties are derived from gradient features and used to terminate the atomistic simulation and bias the atomistic simulation toward unexplored regions of a configurational and/or chemical space of the atomic system. The uncertainty-driven active learning further includes performing active learning to select one or more atomic configurations from the unlabeled atomic data using a trajectory of the atomistic simulation and to train a machine learning model for the interatomic potentials using the one or more atomic configurations. The method can be used in a variety of applications in artificial intelligence (AI) including, but not limited to, several anticipated use cases in medical diagnostics/applications and in healthcare, for example, to optimize predictions or support decision-making.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates an uncertainty-driven active learning (UDAL) approach according to an embodiment of the present invention;

FIG. 2 schematically illustrates the calculation of gradient features used to calculate ensemble-free uncertainties according to an embodiment of the present invention;

FIG. 3 schematically illustrates a batch selection process according to an embodiment of the present invention;

FIG. 4 schematically illustrates the UDAL approach enhanced by inverse material design with automatic differentiation according to an embodiment of the present invention;

FIGS. 5A and 5B provide a schematic overview of AL algorithms for MLIP training, where training structures are selected from data gathered during normal or biased MD simulations according to an embodiment of the present invention;

FIG. 6 is a correlation of maximal atom-based uncertainties with maximal actual atomic force root-mean-squared-errors (RMSEs) according to an embodiment of the present invention;

FIGS. 7A and 7B illustrate a comparison of AL approaches employing biased and unbiased MD simulations to generate the candidate pool of atomic configurations according to an embodiment of the present invention;

FIGS. 8A, 8B, and 8C illustrate an evaluation of the rates of configurational space exploration using biased and unbiased MD simulations employed to generate candidate pools of atomic configurations for AL algorithms;

FIG. 9 illustrates a comparison of ensemble-based to ensemble-free uncertainty-driven approaches for the alanine dipeptide molecule according to an embodiment of the present invention;

FIGS. 10A and 10B illustrate a comparison of naive and uncertainty-driven AL strategies for the MIL-53(Al) system according to an embodiment of the present invention;

FIGS. 11A and 11B illustrate an evaluation of the rates of configurational space exploration using biased and unbiased MD simulations according to an embodiment of the present invention;

FIG. 12 is a block diagram of an exemplary processing system, which can be configured to perform any and all operations disclosed herein;

FIG. 13 illustrates a further comparison of ensemble-based and ensemble-free uncertainty-driven approaches for the MIL-53(Al) system according to an embodiment of the present invention;

FIG. 14 illustrates a position auto-correlation function of MIL-53(Al) system obtained by running biased and unbiased MD simulations at 300 K according to an embodiment of the present invention;

FIG. 15 illustrates an example of a 2-dimensional space partitioning with weights attached to each sub-partition;

FIG. 16 illustrates the structure of the MIL-53(Al) system featuring (a) closed- and (b) large-pore stable phases according to an embodiment of the present invention; and

FIG. 17 is a comparison of AL approaches employing biased and unbiased MD simulations to generate the candidate pool of atomic configurations for the MIL-53(Al) system.

### DETAILED DESCRIPTION

Embodiments of the present invention provide an ensemble-free, uncertainty-driven active learning approach for generating concise, yet comprehensive data sets for training machine learned interatomic potentials. The approach enhances the exploration of configurational and chemical spaces and thereby enhances the generation of uniformly accurate interatomic potentials. Compared to conventional data generation approaches, embodiments of the present invention achieve a speed-up of several orders of magnitude. For example, embodiments of the present invention using the gradient-based approach can outperform conventional data generation approaches in multiple categories, while being more than two times more computationally efficient than approaches using ensemble-based uncertainties. Furthermore, the approach according to embodiments of the present invention achieves a speed-up by an order of magnitude compared to active learning pipelines according to existing technology. Thus, embodiments of the present invention provide machine learning methods and systems with enhanced computer functionality by providing for faster computation with a reduced demand on computational resources, which saves computation power and valuable computational resources, while at the same time generating training data which provides for the modeling of more accurate interatomic potentials of atomic systems. This facilitates the generation of new and improved atomic configurations.

Further, embodiments of the present invention provide solutions to the technical problems of MD simulations, including limited exploration capability, and ensembles, including computational inefficiency, by introducing the ensemble-free uncertainty-driven active learning approach. In the approach, uncertainty-bias drives an atomistic simulation toward unexplored regions of the configurational and chemical spaces. This is achieved by adding a model's uncertainty to potential energy of the systems and computing biased atomic forces by an automatic differentiation technique. To increase the configurational space explored by MD simulations, embodiments of the present invention provide a biasing technique which utilizes bias stresses, which are computed as a gradient of uncertainty with respect to infinitesimal strain deformation, to alter internal pressure of the system. The chemical space is explored by using the so-called alchemical gradients of the uncertainty, i.e., uncertainty gradients with respect to atomic numbers. Further, embodiments of the present invention provide to run multiple MD simulations in parallel and use batch selection algorithms to ensure the generation of maximally diverse training data sets.

The configurational space is defined by atomic positions, in particular coordinates of atoms in three-dimensional (3D) space. The chemical space is defined by the types of atoms and their ratios, e.g., in the Ta—V—Cr—W alloy, one can have different ratios between Ta, V, Cr, and W (or change one of these atoms by some different atom type). Positions can be changed using forces (actual and biased ones) during simulations or alchemical gradients can be used to change the atom types. The composition is changed by changing the relative abundance of atomic types. Parameters of an atomic system are 3D coordinates and respective atomic numbers (types; for example, 1 for hydrogen and 6 for carbon).

In a first aspect, the present disclosure provides a computer-implemented, machine learning method for training interatomic potentials of an atomic system that includes performing uncertainty-driven active learning. The uncertainty-driven active learning includes running an atomistic simulation of the atomic system using ensemble-free uncertainties to generate unlabeled atomic data. The ensemble-free uncertainties are derived from gradient features and used to terminate the atomistic simulation and bias the atomistic simulation toward unexplored regions of a configurational and/or chemical space of the atomic system. The uncertainty-driven active learning further includes performing active learning to select one or more atomic configurations from the unlabeled atomic data using a trajectory of the atomistic simulation and to train a machine learning model for the interatomic potentials using the one or more atomic configurations.

In a second aspect, the present disclosure provides the method according to the first aspect, wherein the ensemble-free uncertainties include atom-based uncertainties calibrated using conformal prediction with root-mean-squared errors (RMSEs) of atomic forces of the atomic system.

In a third aspect, the present disclosure provides the method according to the first or second aspect, wherein the atomistic simulation updates the atomic forces based on the atom-based uncertainties, computes bias forces and updates atomic positions of the atomic system until a stopping criterion is met, at which point the active learning is performed.

In a fourth aspect, the present disclosure provides the method according to any of the first to third aspects, wherein the stopping criterion is reached when the atomic forces of the atomic system with updated atomic positions are predicted to have larger RMSEs than a previous iteration.

In a fifth aspect, the present disclosure provides the method according to any of the first to fourth aspects, wherein the trained machine learning model is used to run a further atomistic simulation for property prediction which does not use uncertainty bias, the property prediction being for an atomic position, an atomic number, a cell parameter or a composition of the atomic system.

In a sixth aspect, the present disclosure provides the method according to any of the first to fifth aspects, further comprising computing gradients of the property prediction using automatic differentiation to update the atomic system, and repeating the uncertainty-driven active learning using the updated atomic system as input.

In a seventh aspect, the present disclosure provides the method according to any of the first to sixth aspects, further comprising running a plurality of further atomistic simulations in parallel with the atomistic simulation, wherein the one or more atomic configurations are selected using a batch selection mechanism based on concatenated trajectories of the atomistic simulations.

In an eighth aspect, the present disclosure provides the method according to any of the first to seventh aspects, wherein the batch selection mechanism uses informativeness, diversity and representativeness as criteria to select the one or more atomic configurations.

In a ninth aspect, the present disclosure provides the method according to any of the first to eighth aspects, wherein alchemical gradients of the ensemble-free uncertainties are computed with respect to atomic numbers and used drive the atomistic simulation to unexplored regions of the chemical space of the atomic system.

In a tenth aspect, the present disclosure provides the method according to any of the first to ninth aspects, wherein gradients of the ensemble-free uncertainties are computed with respect to atomic positions, strain deformation and/or cell parameters and are used to derive bias forces and/or to change an internal pressure of the atomic system and drive the atomistic simulation to unexplored regions of the configurational space of the atomic system.

In an eleventh aspect, the present disclosure provides the method according to any of the first to tenth aspects, wherein the ensemble-free uncertainties are computed from gradients computed for each atom of the atomic system and a total gradient, which is a sum of the gradients computed for each atom of the atomic system.

In a twelfth aspect, the present disclosure provides the method according to any of the first to eleventh aspects, wherein ensemble-free uncertainties include a distance-based uncertainty and a posterior-based uncertainty computed from the gradients, the distance-based uncertainty being computed as a minimum distance from a respective structure to other structures in a space defined by the gradient features, and the posterior-based uncertainty being computed as Gaussian process regression variance using the gradient features.

In a thirteenth aspect, the present disclosure provides the method according to any of the first to twelfth aspects, wherein the gradient features are computed using outputs of a multilayer perceptron, the gradient features possessing a product structure, and wherein a dimensionality of the gradient features is reduced using tensor sketching and/or by using last-layer gradient features.

In a fourteenth aspect, the present disclosure provides a computer system for training interatomic potentials of an atomic system, the computer system comprising one or more hardware processors, which alone or in combination, are configured to provide for execution of uncertainty-driven active learning according to any of the first to thirteenth aspects.

In a fifteenth aspect, the present disclosure provides a tangible, non-transitory computer-readable medium containing instructions for training interatomic potentials of an atomic system, which, upon being executed by one or more processors, provide for execution of uncertainty-driven active learning comprising the following steps according to any of the first to thirteenth aspects.

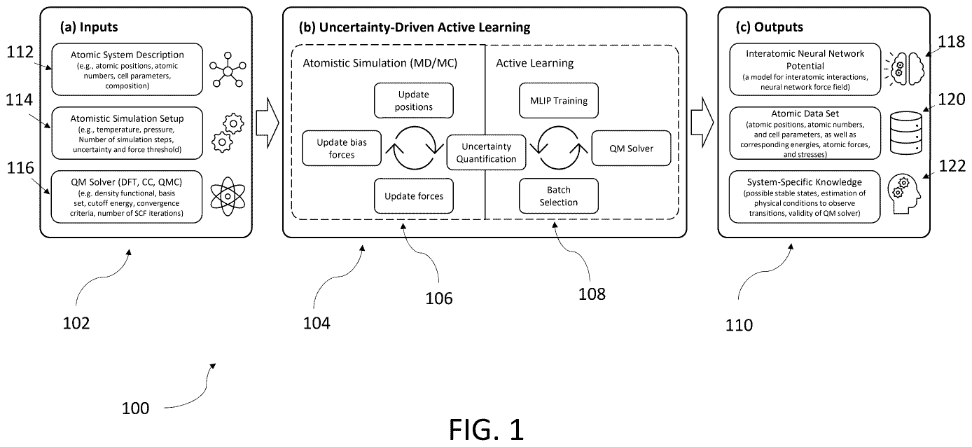

FIG. 1 is a schematic overview of a method and system 100 for uncertainty-driven active learning (UDAL) according to an embodiment of the present invention, in which box 102 shows (a) inputs to the UDAL, box 104 shows (b) a UDAL method comprised of two generic steps 106 and 108: atomistic simulation at step 106 and active learning at step 108, and box 110 shows (c) outputs of the UDAL approach. The two generic steps 106 and 108 of box 104, in an embodiment, can include running an atomistic simulation and active learning.

For example, in step 106, running an atomistic simulation (e.g., MD, MC, etc.) can be performed to generate an unlabeled pool of atomic data, in particular a data pool without reference total energies, atomic forces, and stresses. This step includes uncertainty evaluation, updating atomic forces/stresses, computing bias atomic forces/stresses, and updating atomic positions. Uncertainty evaluation is provided for: (1) estimating whether atomic forces of the updated atomic structure will be predicted with larger root-mean-squared errors (RMSEs) and the atomistic simulation should be terminated (in this case, UDAL moves from atomistic simulation to active learning step); and (2) computing bias atomic forces/stresses to drive the atomistic simulation toward high-uncertainty regions, thereby facilitating exploration of the system's configurational space.

In step 108, running active learning can be performed to select the maximally informative, diverse, and representative atomic configurations and training an MLIP on the updated training data set. The MLIP is a neural network-based model thereby trained to learn interatomic potentials. This step includes uncertainty evaluation, batch selection, evaluation of reference total energies, atomic forces, and stresses, as well as training the new generation of MLIP. The reference values are computed using a quantum-mechanical (QM) solver (e.g., DFT, CC, etc.). The active learning approach according to an embodiment of the present invention employs batch selection algorithms, i.e., selection algorithms which allow selecting multiple atomic configurations from the unlabeled pool at once. Furthermore, using batch selection algorithms, atomic configurations are selected from concatenated trajectories of multiple atomistic simulations run in parallel.

The uncertainty evaluation in steps 106 and 108 can be similar from an algorithmic perspective. The difference is in applying uncertainties during (a) atomistic simulations in step 106 and (b) active learning in step 108. In step 106, the uncertainty used depends on the atomic structure, in particular the 3D coordinates of atoms, to compute bias forces and stresses that drive the simulation toward uncertain regions. Local atom-based uncertainties can also be used to terminate the simulation if they enter unphysical regions. In step 108, uncertainty is used as a measure of informativeness to choose atomic structures, which will improve the performance of the MLIP and compute labels for selected structures.

Box 102 of FIG. 1 depicts the inputs to the UDAL approach. Input 112 is an atomic system (e.g., a molecule, bulk solid or liquid, etc.), which can be defined by several parameters: (1) composition, i.e., the relative number of atomic species (elements); (2) atomic numbers, i.e., atomic species (elements); (3) atomic positions, i.e., the relative positions of atoms; (4) cell parameters, i.e., lengths of and angles between principal axes of the unit cell; (5) total or atomic charges of the system, etc. Input 114 can include an atomistic simulation setup. The setup for the atomistic simulation can be defined by the physical conditions and simulation methods: (1) a statistical ensemble (microcanonical ensemble (NVE), canonical ensemble (NVT), or isothermal-isobaric ensemble (NpT)) employed to run the atomistic simulation, e.g., an MD, MC or Hamiltonian Monte Carlo (HMC) simulation; (2) a length of the atomistic simulation, e.g., the number of MD timesteps; (3) for MD simulations, the integration step has to be defined; (4) defining an uncertainty method to bias the atomistic simulation, e.g., bias forces and stresses, and (5) an uncertainty threshold to terminate it before highly distorted, unphysical configurations have been explored. Input 116 can include a QM solver defined to evaluate reference level of theory, providing reference energies, atomic forces, stresses, etc. for training MLIPs. The respective setup can be different depending on the modelled system. The approach according to embodiments of the present invention does not restrict the selection of reference QM Solvers. Some examples for DFT-based solvers include: (1) defining exchange-correlation functionals and suitable basis sets; (2) energy cutoff, the number of self-consistent-field iteration, etc.

Box 110 of FIG. 1 depicts the outputs of the UDAL approach. One of the main outputs of the UDAL approach is an MLIP, such as output 118 of an interatomic potential (e.g., an interatomic neural network potential in a case that the MLIP uses a neural network), which models interatomic interactions accurately across the entire configurational space of interest. In other words, embodiments of the present invention enable to provide an MLIP which is accurate at all physical conditions that are interesting to investigate a specific atomic system. Another output of the UDAL approach can include output 120 of an atomic data set. Generated high-quality training data sets is another output of the pipeline according to embodiments of the present invention, which represents a huge improvement to computational functionality, as well as to the field of machine learning training for interatomic potentials as very few high-quality molecular and materials data bases exist. Embodiments of the present invention allow exploring the whole material class by sequentially or in parallel building the data sets. Another output of the UDAL approach can include output 122 of system-specific knowledge. Generated atomic system-specific knowledge can include uncertainty-driven dynamics, which explores all stables states of the system, including possible transitions. For example, embodiments of the present invention allow to construct reactive potentials and, thus construct reaction networks, necessary to investigate complex chemical processes.

### Technical Process 1: Ensemble-Free Uncertainties

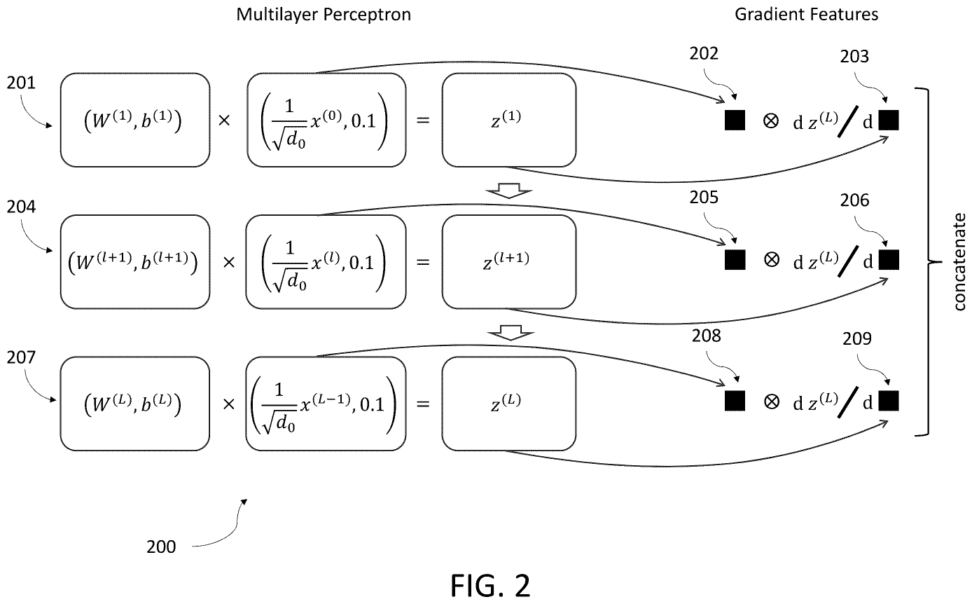

FIG. 2 illustrates a schematic overview of a method and system 200 for determining gradient features used to calculate ensemble-free uncertainties according to an embodiment of the present invention. Gradient features of a neural network (NN)-based interatomic potential possess a product structure, simplifying their calculation using automatic differentiation (AD). The product structure means the gradient of the output (z^{(L)}) of a neural network with respect to its parameters 201, 204, 207 and can be written as a product of inputs 202, 205, 208 to each hidden layer and the gradients of the output with respect to activations 203, 206, 209 of each hidden layer. This fact simplifies the AD calculation as it is now only needed to compute only

$$\frac{d {z}^{( L )}}{d {z}^{( l )}} ⊗ {x}^{( l - 1 )}$$

and concatenate them instead of the high-dimensional

$$\frac{d {z}^{( L )}}{d θ} ,$$

where θ are all parameters of the network (see also discussion of sketched gradient features below). FIG. 2 shows the calculation of gradients of the output of a multilayer perceptron with respect to its parameters. Each line/row in FIG. 2 shows that the calculation of

$$\frac{d {z}^{( L )}}{d θ} ,$$

where θ are all network parameters, is split into a separate calculation for each layer. The results are then concatenated, thereby being combined into one high-dimensional vector. W^{(l)} is the weight matrix of layer l, b^{(l)} is the bias vector of layer l, d indicates an ordinary derivative, x^{(l)} are inputs to layer l+1, z^{(l)} are activations of layer l, l is the index running over the number of layer in the multilayer perceptron, and L is the maximal number of layers in the multilayer perceptron.

Embodiments of the present invention overcome the technical problem of uncertainty quantification, and in particular provide to improve its accuracy to allow to better (1) terminate atomistic simulations before exploring highly distorted, unphysical atomic configurations, and (2) drive atomistic simulations toward unexplored regions, facilitating exploration of the configurational space of interest. Further, embodiments of the present invention overcome the technical problem of an ensemble of MLIPs, which is typically used for uncertainty quantification, making online learning approaches computationally inefficient and prohibiting their application in domains such as material and drug discovery. In particular, the approach according to embodiments of the present invention provide for ensemble-free uncertainty quantification with improved accuracy and computational efficiency.

According to embodiments of the present invention, ensemble-free uncertainties are derived from gradient features to (1) terminate atomistic simulations, and (2) to bias them toward high-uncertainty regions of the configurational space. Gradient features for NN-based interatomic potentials are computed as gradients of the output of the respective multilayer perceptron with respect to its parameters. As most of the NN-based interatomic potentials assume the locality of interatomic interaction, gradient features are obtained as a sum of atomic gradient features. The gradients of the output of the multilayer perceptron, which have a product structure as described above, are computed with respect to its parameters, in particular weights and biases.

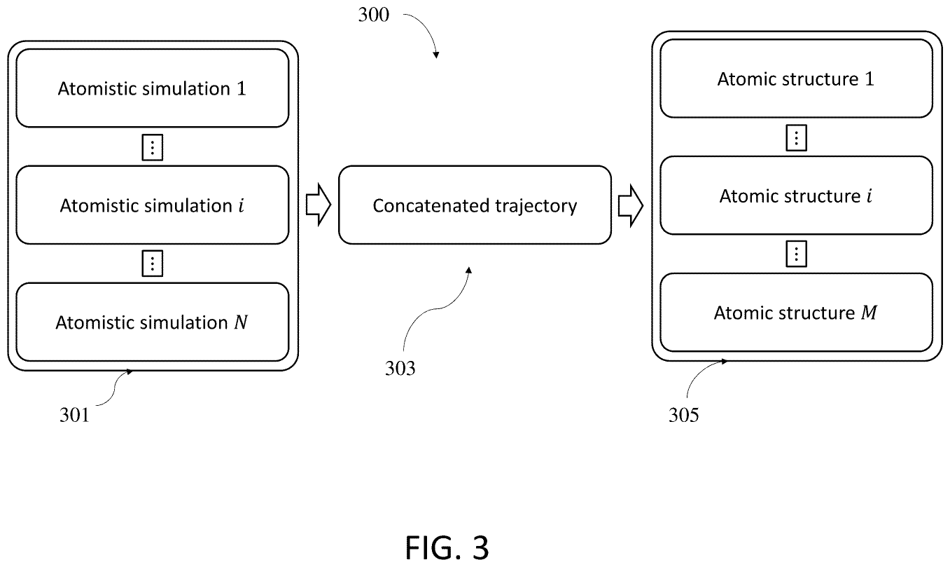

For example, FIG. 3 schematically illustrates a batch selection allowing for running multiple parallel atomistic simulations and simultaneous selection of maximally diverse atomic configurations from the concatenated trajectory according to an embodiment of the present invention. The concatenated trajectory is obtained by appending or stacking together trajectories acquired during multiple parallel simulations. The maximally diverse atomic configurations are chosen by either maximizing the distance between selected configurations in the space spanned by the gradient features (gradients of the multilayer perceptron evaluated for the respective structures) or by maximizing the determinant of the covariance matrix (Gaussian process regression) built using gradient features (see also discussion of batch selection methods below). Using automatic differentiation, the atomic gradient features are computed and then the atomic gradient features are summed to obtain gradient features that describe the whole atomic system. In an embodiment, the present invention uses both atomic gradient features and their sum to define atom-based uncertainties and uncertainties of the whole system, respectively. For the automatic differentiation, a PyTorch implementation of automatic differentiation can be used, or other implementations of automatic differentiation used in existing machine learning technology for training models can also be used. Automatic differentiation uses computational graphs to compute the derivatives of a function with respect to its inputs, using either forward or reverse mode. It records and propagates intermediate values and gradients through the graph.

Product structure of gradient features simplifies their calculation. By using random projections (which can also be referred to as sketching), the dimensionality of gradient features is reduced, thereby improving the computational efficiency of UDAL. For example, a tensor sketching technique according to an embodiment of the present invention is as follows: (1) define a random matrix U of size Nᵣₚ×N_{feat} with Nᵣₚ denoting the number of random projections and N_{feat} being the length of the gradient feature vector φ, (2) compute φᵣₚ=U·φ, which has only Nᵣₚ dimensions, which is much lower than N_{feat}, and (3) tensor sketching by adding contributions from different layers of the multilayer perceptron instead of concatenating them (see also discussion of sketched gradient features below). This technique is used to reduce the dimensionality of gradient features after the gradient features have been computed with automatic differentiation.

Embodiments of the present invention use gradient features to quantify uncertainty of the whole atomic system and each atom separately. The uncertainty of the whole atomic system can be identified as total uncertainty, while the atom-based uncertainty can be calibrated using conformal prediction (CP) with RMSEs in atomic forces, thereby providing a sensitive metric for identifying the onset of high-uncertainty regions. Using CP with RMSEs to calibrate the atom-based uncertainty can be performed in an embodiment of the present invention as follows: (1) choose a calibration data set, the validation data set used for early stopping, (2) compute force RMSEs and atom-based uncertainties for the calibration data set, (3) compute the ratio (force RMSEs)/(atom-based uncertainties) and a quantile defined by the hyperparameter of CP referred to as confidence level. The confidence level defines the probability of atom-based uncertainties not being lower than the force RMSE (see also discussion of conformal prediction below). The same rules hold for calibrating total uncertainty, except that energy RMSE and total uncertainty are computed instead. One or both the total and atom-based uncertainties are used to bias the system toward unexplored regions of the configurational space. For total uncertainties, the dynamics are biased with their gradients with respect to the inputs, in particular atomic positions and/or cell parameters. For atom-based uncertainties, these are first summed and then the same procedure as for total uncertainties can be followed to bias the dynamics.

Technical Process 2: Uncertainty Gradients with Automatic Differentiation

Embodiments of the present invention overcome the technical problems for how to drive atomistic simulations toward unexplored regions, which requires calculation of uncertainty gradients with respect to parameters that define the configurational space of interest. Analytic derivatives restrict the applicability of such techniques to using ensemble-based uncertainty quantification and interatomic forces for biasing, e.g., an MD simulation.

Embodiments of the present invention overcome these technical problems by using AD to compute uncertainty gradients with respect to the parameters of an atomic system (e.g., gradients with respect to atomic numbers, atomic positions, and strain deformations). Using AD, embodiments of the present invention enable to drive an atomistic simulation toward any unexplored region of configurational and chemical spaces. Particularly, the chemical space can be explored by the so-called alchemical gradients of the energy. Embodiments of the present invention compute alchemical gradients as uncertainty gradients with respect to atomic numbers

$$( \frac{d u}{d Z} ,$$

where u is the uncertainty and Z is the atomic number/type) using AD as follows: (1) compute uncertainties using gradient features, and (2) compute the alchemical gradients of the uncertainties using AD. For the alchemical gradients, it is possible to run optimization algorithms that change the atom types to maximize the uncertainty. Furthermore, embodiments of the present invention compute, e.g., uncertainty gradients with respect to an infinitesimal strain deformation, which provides bias stress that facilitates exploration of pressure induced configurational changes by reducing the internal pressure of the atomic bulk system. For bias stress, the gradient of the uncertainty is computed with respect to strain deformations

$$( \frac{d u}{d ε} ,$$

evaluated for ε=0). Internal pressure is defined as the energy gradient with respect to ε:

$$T r ( \frac{d E}{d ε} ) / 3$$

evaluated for ε=0. These are added:

$$- Tr ( \frac{d u}{d ε} ) / 3 + T r ( \frac{d E}{d ε} ) / 3 ,$$

which physically leads to the former acting against the latter, reducing the internal pressure. Here, Tr means trace of a matrix (sum of matrix elements on the main diagonal of this matrix), E is the energy, and ε is the infinitesimal strain deformation. Thus, gradients with respect to atomic positions and strain deformations/cell parameters are used to explore configurational space and the alchemical gradients (gradients with respect to atomic numbers) are used to explore chemical space.

Thus, in the Technical Process 1, gradients of the output of a multilayer perceptron are computed with respect to its parameters (weights and biases) whereas here, in the Technical Process 2, gradients of the resulting uncertainties are computed with respect to atomic coordinates to get bias forces, stresses, etc. Automatic differentiation is used for each of these processes. The uncertainties include: (1) distance-based uncertainty, which is computed as the minimum distance from a structure (e.g., observed during MD or taken from a different data set) to all structures in the training data set in the space defined by the gradient features (evaluated for respective structures) computed earlier, and (2) posterior-based uncertainty, which computes posterior (Gaussian process regression) uncertainty (variance) using gradient features of the given structure and those in the training data set (see also the discussion of gradient-based uncertainties below). These uncertainties are used to bias the dynamics of the systems with gradients of these uncertainties computed with respect to atomic positions, strain deformations and/or cell parameters. These gradients are added to actual physical forces and stresses to modify Newton's equations of motion (see also the discussion of uncertainty-biased molecular dynamics below) and/or to change internal pressure of the atomic system. The uncertainty gradients are used to increase the level of uncertainty of explored structure and bias simulations toward uncertain regions. They essentially do the steepest ascent in the uncertain domain to maximize the uncertainty of structures explored during dynamics.

### Technical Process 3: Batch Selection for Maximally Diverse Data Sets and Parallel Atomistic Simulations

Embodiments of the present invention overcome the technical problems of existing selection methods that are used in a setting with multiple parallel atomistic simulations, which leads to selecting multiple correlated atomic configurations, deteriorating the performance of MLIPs. An alternative is running a single atomistic simulation and selecting a single configuration per iteration, in an iterative learning approach such as UDAL.

Embodiments of the present invention overcome these technical problems by using batch selection algorithms for approaches such as UDAL, which generate unlabeled pools of atomic data dynamically during high-throughput atomistic simulations. As a result, scheme 300 allows for parallel simulations 301, including the atomistic simulation 1, the i-th atomistic simulation, and the N-th atomistic simulation to be run in parallel. For example, running multiple parallel MD simulations is computationally more efficient than running a long MD simulation. Additionally, parallel MD simulations explore larger portions of configurational space during a single iteration of UDAL. Alternatively, parallel MD simulations allow simultaneous exploration of configurational spaces for various materials. Furthermore, embodiments of the present invention constrain diversity for the selected batch of atomic configurations. Given that parallel MD simulations provide highly correlated structures, this constraint advantageously provides to generate maximally diverse training data sets which do not deteriorate the performance of final MLIPs. Referring to the process 300 of FIG. 3, an embodiment of the present invention can then employ batch selection algorithms, e.g., selection algorithms which allow selecting multiple atomic configurations 305 from a concatenated trajectory 303 from multiple atomistic simulations 301 run in parallel. A batch of atomic configurations 305 can thereby be simultaneously chosen from the concatenated trajectory 303, such as maximally diverse atomic configurations (chosen by either maximizing the distance between selected configurations in the space spanned by the gradient features (gradients of the multilayer perceptron evaluated for the respective structures) or by maximizing the determinant of the covariance matrix (Gaussian process regression) built using gradient features), fed into a DFT solver, and used to generate energies and atomic forces that update the training data set of atomic structures.

Technical Process 4: Inverse Material Design with Automatic Differentiation

Embodiments of the present invention address the technically challenging task of finding compositions or crystal structures of materials which should possess certain mechanical, thermodynamics, or electrical properties in a computationally efficient manner. For example, iterating through all possible elements and their relative abundance is a combinatorial task, and thus is infeasible to solve on a standard compute node.

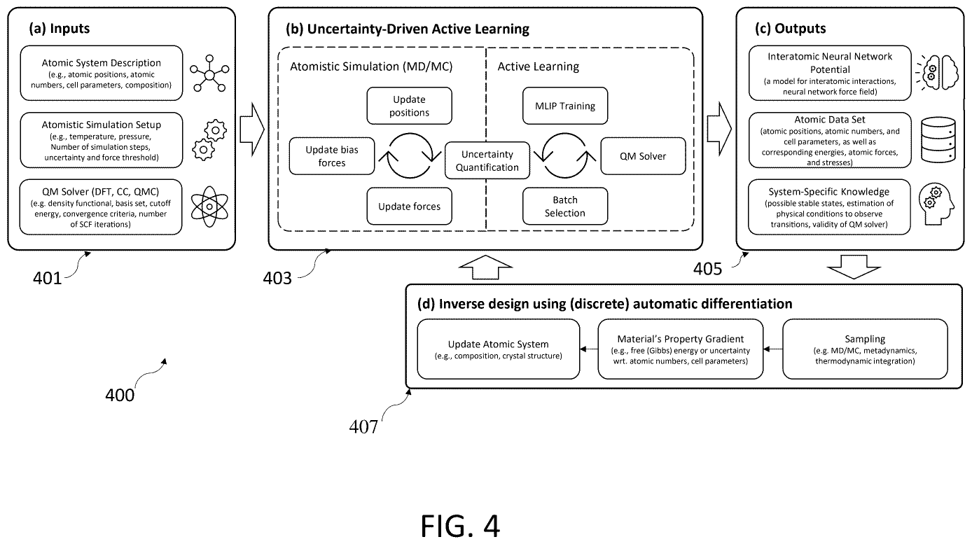

Embodiments of the present invention overcome these technical challenges by using automatic differentiation for the inverse design of molecular and material systems and enhance the UDAL approach with it, as shown in FIG. 4. For example, a process for using the automatic differentiation here according to an embodiment of the present invention includes: (1) running simulations and obtaining a distribution of energies, (2) using this distribution, computing a property of a material, and (3) calculating this property within a fully differentiable framework, allowing to compute its gradient with respect to atomic positions, atomic numbers, etc. using automatic differentiation, and (4) optimizing using these gradients to find the optimal property. Embodiments of the present invention use highly accurate MLIPs generated by the UDAL approach for running, e.g., long MD simulations to compute thermodynamic or mechanical properties of the investigated atomic system. In other words, embodiments of the present invention output a highly accurate MLIP, which can then be used be used to run simulations that accurately predict the properties of molecules and materials. Performing these simulations in a fully differentiable framework allows to compute gradients of the estimated properties with respect to the parameters of the atomic system such as, e.g., atomic numbers or cell parameters. For this purpose, embodiments of the present invention use an automatic differentiation technique. This approach allows to alter the atomic system in a data-driven manner toward optimized performance in terms of the estimated property.

For example, as shown in the embodiment of the process 400 of FIG. 4, (a) inputs are provided to a UDAL approach in step 401. The UDAL approach is performed in step 403 including running atomistic simulations and performing active learning, to provide (c) outputs at step 405. Step 407 provides (d) inverse design using (discrete) automatic differentiation that updates the atomic system provided as input to the UDAL approach. During the inverse design process in step 407, the outputs of step 405 can be sampled and the property gradients can be determined.

Alternatively, derivatives over the accumulated uncertainty through the atomistic simulation can be calculated. By doing so, the UDAL approach can be forced to explore parts of the chemical space of a material class not well represented by the current training data.

Advantageously, the inversed design according to an embodiment of the present invention can be used in combination with every enhanced sampling technique, designed for free energy calculation, as well as other properties which can be derived from it. For example, one can use it together with thermodynamic integration, metadynamics, or Hamiltonian Monte Carlo.

Embodiments of the present invention thus provide for general improvements to computers in machine learning systems by each of the technical processes 1-4, which provide for computational improvements and overcoming technical problems both individually and in combination as discussed above. Moreover, embodiments of the present invention can be practically applied to use cases to effect further improvements in a number of technical fields including, but not limited to, medical (e.g., digital medicine, personalized healthcare, AI-assisted drug or vaccine development, etc.), material design, and energy production.

### Exemplary Application Embodiment 1: Modelling Molten Salts for Next Generation Energy Production and Storage Materials

Molten salts play an important role in various technical applications including electrolysis-based metal production, fuel processing for the next-generation nuclear reactor, concentrated solar power, and liquid metal battery applications. Thus, molten salts hold the promise to be a fundamental technology in fields such as energy storage and rare earth element recycling, e.g., pyro-processing of nuclear waste. For investigating thermodynamic properties of molten salts, such as solubility of rare earth elements or reaction electrode potentials in them, long MD simulations are necessary. Furthermore, highly accurate and, thus, computationally inefficient QM solvers are required, to provide accurate estimates of the above properties.

In this exemplary application, an embodiment of the present invention overcomes these technical problems using the UDAL approach to generate high-quality training data sets for training MLIPs. The UDAL approach allows to investigate molten salts of interest in a data-driven manner, without requiring prior knowledge of physical conditions (e.g., temperature and pressure) at which conformational changes occur. The UDAL approach utilizes uncertainty-driven atomistic simulations, which guarantees that generated MLIPs are accurate across the relevant configurational space.

In this exemplary application, all of the technical processes 1-4 can be used. Particularly, inverse design provides the means of iterating through compositions or box sizes to generate potentials and training data sets which perform best on the defined problem.

The inputs for this exemplary application are initial compositions of molten salts. Additionally, configurations with rare earth elements dissolved in molten salts can be used for input. All initial configurations are provided as a geometry with atomic numbers, atomic positions, and cell parameters. Atomistic simulations, in particular MD simulations, are performed at constant pressure, i.e., NpT statistical ensemble. The integration time step is set to values larger than 2 fs, while simulation temperature can be fixed at higher values, if known. Similarly, external pressure is fixed, if suitable values are known. The uncertainty method and the respective uncertainty threshold values are set up, keeping the exploration to well-behaving regions in terms of predicted energies and forces. Embodiments of the present invention use uncertainty-driving to enhance exploration independent of chosen physical conditions, and define the biasing strength to dynamically adjust the portion of bias forces/stresses.

The outputs for this exemplary application are MLIPs describing interactions within molten salts and between molten salt ions and rare earth elements, and a data base including atomic configurations with respective reference energies, atomic forces, and stresses. Optimized parameters (e.g., composition, cell parameters) of molten salts for desired performance on properties of interest can also be output, as well as a list of possible stable states/phases of investigated molten salts, providing additional knowledge for the design of materials with improved properties. The output of the MLIP or a longer MD simulation using the MLIP can be used to automatically provide molten salt candidates or to control equipment of a production or testing facility to produce the molten salt candidates.

### Exemplary Application Embodiment 2: Modelling Polymers for Next Generation Plastics with Improved Thermodynamic and Mechanical Properties

Polymers play an essential role in nature and technical applications due to their long chain molecular structure. As a variety of monomers (elementary building blocks) exist, they can be used to tune polymers toward the desired property. In this case, tuning chemical composition of the macromolecule as well as its spatial arrangement is a key technical challenge. Furthermore, modelling polymers at quantum chemical accuracy, although technically challenging, can provide for a number of improvements in material design. Large system sizes are prohibitive in this case, limiting quantum chemical calculations to individual polymer building blocks (monomers and oligomers). However, properties of polymers should be computed at mesoscopic scale.

In this exemplary application, an embodiment of the present invention overcomes these technical problems using the UDAL approach to generate highly accurate and computationally efficient MLIPs which scale to necessary system sizes. Thus, an embodiment of the present invention provides the means of investigating thermodynamic and mechanical properties of polymers. Using the inversed design, an embodiment of the present invention is able to change the chemical composition of polymers and, thus, change their properties toward the desired behavior. Changing cell parameters using the inversed design is used to find the optimal values necessary for accurate property calculation.

The inputs in this exemplary application include polymer building blocks, monomers and oligomers, with respective atomic positions and atomic numbers that are provided to the UDAL approach. For the case of bulk materials, periodic cell is used. Atomistic simulations, e.g., MD simulations, are performed at constant volume (NVT statistical ensemble) and constant pressure (NpT statistical ensemble) for molecular and bulk atomic systems, respectively. Small integration timesteps of, e.g., 0.5 fs can be used. The temperature should be set just below the degradation temperature, if known. The uncertainty method and the respective uncertainty threshold values are set up, keeping the exploration to well-behaving regions in terms of predicted energies and forces. Embodiments of the present invention use uncertainty-driving to enhance exploration independent of chosen physical conditions, and define the biasing strength to dynamically adjust the portion of bias forces/stresses.

The outputs for this exemplary application are MLIPs describing inter- and intraatomic interactions within polymers at the chosen level of theory. They allow to scale the system size suitable for the property calculation. The UDAL approach produces a data base covering versatile class of functional monomers and oligomers used to build polymers with desired properties. From the generated data sets insight into conformational changes occurring during MD simulation are gained, to identify most stable atomic configurations. The output of the MLIP or longer MD simulation can be used to automatically provide potential polymeric material composition candidates or to control equipment of a production or testing facility to produce the polymeric materials.

### Exemplary Application Embodiment 3: Modelling Sustainable Materials for Renewable Energy Production Using Water Splitting

Hydrogen gas is a renewable energy carrier which promises to supply the world's energy demand. Furthermore, it can reduce greenhouse gas emission. Hydrogen gas can be produced by electrochemical water splitting (e.g., fuel cells). However, water splitting is extremely energy intensive and requires very harsh acidic conditions. Only a few materials appear to be stable and active at these conditions. Thus, identifying possible cheap material candidates is a key technical challenge, requiring extensive exploration of configurational and chemical spaces. Another example is splitting the seawater. In this case, competing catalytic reactions are observed, while environmental conditions lead to the degradation of the catalyst.

In this exemplary application, an embodiment of the present invention overcomes these technical problems using the UDAL approach, which is not restricted to non-reactive chemistry, to generate MLIPs accurate for both modelling chemical reactions with high accuracy and designing catalytic materials which stay stable for longer time at harsh environmental conditions. Furthermore, the inversed design according to an embodiment of the present invention allows to investigate influence of chemical compositions of catalyst material toward optimal reaction rate constants, binding energy, etc.

The inputs in this exemplary application include initial (e.g., equilibrium) atomic structures of water and hydrogen molecules, as well as OH radicals, put on top of the material surface to the UDAL approach. It may include different surface orientations. Various atomistic simulations are set up to explore reactions and surface effect on them. For example, to obtain reaction paths and the corresponding energy barriers, the following atomistic simulations are used: geometry optimization, transition state, and minimum energy path search. To investigate environmental influence and catalytic activity of different materials, MD simulations are run at constant volume (NVT statistical ensemble). The uncertainty method and the respective uncertainty threshold values are set up, keeping the exploration to well-behaving regions in terms of predicted energies and forces. Embodiments of the present invention use uncertainty-driving to enhance exploration independent of chosen physical conditions, and define the biasing strength to dynamically adjust the portion of bias forces/stresses.

The outputs in this exemplary application are trained reactive MLIPs which can be used to run longer MD simulations, while breaking and establishing bonds between atoms (reactive MD). The generated training data contains a network of reactions, including possible reaction paths. Different compositions of catalytic materials having different activity and stability for given physical and environmental conditions can also be output for improved material design. The output of the MLIP or longer MD simulation can be used to automatically provide potential material candidates or to control equipment of a laboratory.

### Exemplary Application Embodiment 4: AI Assisted Drug Design and/or Modelling Small Drugs—Interaction and Binding Properties

Binding (free) energy is essential for drug design, as well prediction of other properties, as toxicity or other production related property as stability and production process.

In this exemplary application, an embodiment of the present invention can be used to learn the MLIP for modelling the binding site, and if possible, of the whole protein and molecule. A special method is provided to model coarse-grained interaction, when coarse-grained is used during simulation.

The inputs in this exemplary embodiment include molecular descriptions of the elements to be simulated and functions to derive the properties from the measurements, as well as a method to simulate with the learned potentials.

The outputs in this exemplary embodiment are an MLIP that will be used for the simulation of the properties. The output of the MLIP or longer MD simulation can be used to automatically provide potential drug design candidates or to control equipment of a wet lab.

In an embodiment, the present invention provides a method for generating complete yet minimal data sets for training interatomic neural network potentials, the method comprising the steps of:

1) System set up, which includes the definition of the atomic system, the QM solver and the model to train:
a. Choosing an uncertainty quantification method for driving an atomistic simulation toward unexplored regions. For the uncertainty quantification, two ensemble-free uncertainties could be used: distance-based and posterior-based. An uncertainty estimated with a MLIP ensemble, or another differentiable uncertainty could also be used. This step includes defining parameters of the atomic system (atomic positions, atomic numbers, strain deformation, etc.) with respect to which gradients of the uncertainty are computed using automatic differentiation to bias the atomistic simulations.
b. Defining or choosing a neural network architecture (e.g., MLIP), including local atomic representation, to model inter- and intra-atomic interactions. The selected architecture should be sufficient to model the relevant ranges of interaction in the investigated atomic system. Advantageously, embodiments of the present invention are architecture agnostic, such that the selection of the neural network architecture can be considered a design choice.
c. Choosing an atomistic simulation (e.g., MD) which can be used to explore the accessible configurational and chemical spaces, combined with the uncertainty-bias. This step includes, e.g., identifying the statistical ensemble in which the respective atomistic simulation is performed. Other parameters can be MD time step or length of the chosen atomistic simulation. Furthermore, an uncertainty threshold value is defined to stay in the physically reasonable regions over the course of simulation. The threshold can be determined based on the type of atomic bonds (covalent, ionic, metallic, etc.), and the desired accuracy of MLIPs. Typical values are between 0.5 and 1.5 eV/Ang. for atomic forces/atom-based uncertainty.
d. Choosing a quantum mechanical solver (e.g. DFT) suitable for the atomic system. Like the determination of the threshold value, the choice may depend on the type of atomic bond and the desired accuracy. Also, one may perform benchmarks, comparing some observables to experiments or more accurate quantum mechanical solvers. The choice is system-dependent. The quantum mechanical solver is used to provide accurate reference values for energies, atomic forces, and stresses while training MLIPs. This step includes defining, e.g., exchange-correlation functionals, basis sets, etc.

2) Running an iterative learning process of UDAL, until the maximal desired data set size or accuracy of the interatomic potential evaluated as RMSEs in energies, atomic forces, and stresses has been reached. The parameters of intermediate and the final MLIPs are stored to perform large scale atomistic simulations for property prediction. The AL calls the DFT on the selected batch of samples. This batch of samples is selected from trajectories obtained by running uncertainty-biased atomistic simulations with intermediate MLIPs.

3) In some embodiments, using the highly accurate MLIP trained with UDAL, running large scale molecular dynamics simulations for property prediction. These simulations do not use uncertainty bias.

4) In some embodiments, in case of inverse design:
a. Run large scale, fully differentiable atomistic simulations to, e.g., sample energies from the Boltzmann distribution, using the highly accurate MLIP trained with UDAL to obtain an estimated property.
b. Compute gradients of the estimated property, such as free energy or isobaric thermal capacity derived from it, with respect to parameters of the atomic system, such as atomic numbers or cell parameters.
c. Update atomic system to provide a new input to UDAL.

Embodiments of the present invention provide for the following improvements and technical advantages over existing technology:

1) Providing to use ensemble-free uncertainties (what) for terminating atomistic simulations and driving them toward unexplored, high-uncertainty regions (how). Sketched or last-layer gradient features are used to compute these uncertainties. For terminating atomistic simulations, embodiments of the present invention provide to use atom-based, ensemble-free uncertainties, calibrated with atomic forces RMSE using conformal prediction.
a. With batch selection using three criteria (representativeness, diversity and informativeness). For example:
Informativeness: Use only uncertainty to choose structures. This criterion might lead to selecting similar structures.
Informativeness+Diversity: Here, uncertainty is used and it is ensured that structures are not similar. As mentioned above, the maximally diverse atomic configurations are chosen by either maximizing the distance between selected configurations in the space spanned by the gradient features (gradients of the multilayer perceptron evaluated for the respective structures) or by maximizing the determinant of the covariance matrix (Gaussian process regression) built using gradient features.
Informativeness+Diversity+Representativeness: Here, in addition to previous points, the data distribution is taken into account, meaning to select more points from denser populated regions of the space, spanned by the gradient features.
b. Use of calibration of the uncertainty for transfer of method across atomic systems.

2) Providing to use automatic differentiation (how) for computing uncertainty gradients (what) with respect to any parameters (how) of an atomic system which defines its configurational or chemical space. These parameters can be discrete, e.g., atomic numbers, or continuous, e.g., atomic positions and cell parameters. Embodiments of the present invention provide to derive bias stress by computing uncertainty gradients with respect to infinitesimal strain deformation. The derived bias stress is used to reduce internal pressure of a material, facilitating exploration of phase transitions within, e.g., MD simulation in the NpT ensemble at zero external pressure.

3) Providing to use automatic differentiation (how) for computing gradients of material properties (what) obtained through fully differentiable atomistic simulations. These gradients, e.g., with respect to atomic numbers, are used to update the atomic system (aka inverse design) by, e.g., changing the chemical composition.

Compared to conventional approaches used to generate atomistic data sets, which employ the so-called ab-initio MD simulations, embodiments of the present invention provide to achieve a speedup of a factor of several thousands, depending on the system size and the QM solver involved. Furthermore, conventional pipelines require knowledge of the system and technique-specific parameters required to explore the relevant configurational space, e.g., collective variables in a metadynamics simulation. The UDAL according to embodiments of the present invention provides to overcome these technical limitations, as uncertainty-driven atomistic simulations explore the respective configurational space in a data-driven manner, not requiring any prior knowledge. For example, unlike existing technology for training MLIPs, it is not necessary to know the temperatures and pressures at which configurational changes occur according to an embodiment of the present invention.

Further, using ensemble-free uncertainties in UDAL according to embodiments of the present invention allows to speed-up training set generation by about N-times compared to approached using ensembles, where N is the number of models in the ensemble being compared to. Furthermore, the UDAL approach according to embodiments of the present invention speed-up exploration of the configurational space of interest by a factor of 2-4 by effectively reducing the autocorrelation times in position and uncertainty domains, making the atomistic simulations according to embodiments of the present invention explore new points in the respective domains. Thus, relative to existing technology using ensembles, embodiments of the present invention enable a computational time speed-up of 2*N-4*N. Therefore, compared to the smallest possible ensemble of three models used for uncertainty quantification, the speed-up which can be achieved by the UDAL approach is 6-12 times compared to existing technology. In addition to being more computationally efficient, the approach allows to obtain MLIPs with the same or better performance in terms of RMSEs in total energies, atomic forces, and stresses.

Embodiments of the present invention also provide for using automatic differentiation, which allows designing alternative biasing techniques as, e.g., introducing bias stress which alters the internal pressure of the system and drives an atomistic simulation toward exploration of pressure-induces phase transitions. Alternatively, alchemical uncertainty gradients, i.e., uncertainty gradients with respect to atomic numbers, are used to explore the accessible chemical space.

In the following, further background and description of exemplary embodiments of the present invention, which may overlap with some of the information provided above, are provided in further detail, along with empirical data evidencing the enhancements to computer functionality in machine learning systems. To the extent the terminology used to describe the following embodiments may differ from the terminology used to describe the preceding embodiments, a person having skill in the art would understand that certain terms correspond to one another in the different embodiments. Features described below can be combined with features described above in various embodiments.

Machine learning can transform how to model interatomic interactions in molecular and bulk systems. Yet, creating a minimal, complete data set for training interatomic potentials can still remain a challenge. Embodiments of the present invention can provide an ensemble-free active learning approach, using sketched gradient features and MD simulations driven toward uncertain regions.

Uncertainty-driven MD can effectively explore configurational space. However, compared to conventional enhanced sampling techniques that focus on uniform phase-space coverage, uncertainty-driven MD aims at uniformly reducing uncertainty associated with the actual error. In embodiments using alanine dipeptide, uncertainty-driven MD at 300K can explore the entire collective variable space, achieving energy and atomic forces RMSEs comparable to unbiased 1200K simulations. Ensemble-free uncertainties can be combined with automatic differentiation to develop a novel technique for exploring pressure-induced transitions in bulk atomic systems. Applied to MIL-53(Al), embodiments of the present invention can provide methods that explore the two stable states of this flexible metal-organic framework. Employing volume-dependent RMSEs for energies, atomic forces, and stresses, uncertainty-driven active learning approaches can produce uniformly accurate interatomic potentials and surpasses conventional methods like metadynamics and high-pressure MD simulations. With improved computational efficiency and similar or better accuracy compared to ensemble-based methods, embodiment of the present invention can offer a promising alternative for developing interatomic potentials for complex chemical systems.

MLIPs can achieve significant success in modeling interatomic interactions in materials and molecular systems. Their effectiveness can be based on well-parameterized potentials that faithfully reproduce underlying physics, given sufficient training data covering the relevant configurational and chemical spaces as well as an appropriate representation of the atomic system. A wide range of machine learning approaches can be used, including linear and kernel-based models, Gaussian approximation, and NN interatomic potentials, all of which can demonstrate remarkable success in atomistic simulations.

An open technical challenge is the generation of informative yet minimal training data sets without any prior knowledge of the atomic system. Running ab-initio molecular dynamics (MD) simulations using density-functional theory (DFT) to approach this objective can be computationally demanding. Thus, a combination of MLIP driven MD simulations with active learning (AL) to dynamically explore the relevant configurational space can be used instead. The MLIP is iteratively refined during AL. However, covering the phase space of interest might require running longer MD simulations while also defining physical conditions at which relevant conformational changes occur. Alternatively, more advanced approaches for developing training data sets may rely on enhanced sampling techniques, such as introducing a history dependent bias to the potential energy to accelerate phase space exploration. These approaches might require the definition of collective variables (CVs) along which the dynamics is effectively biased, which limits their application to the configurational space that may already be known.

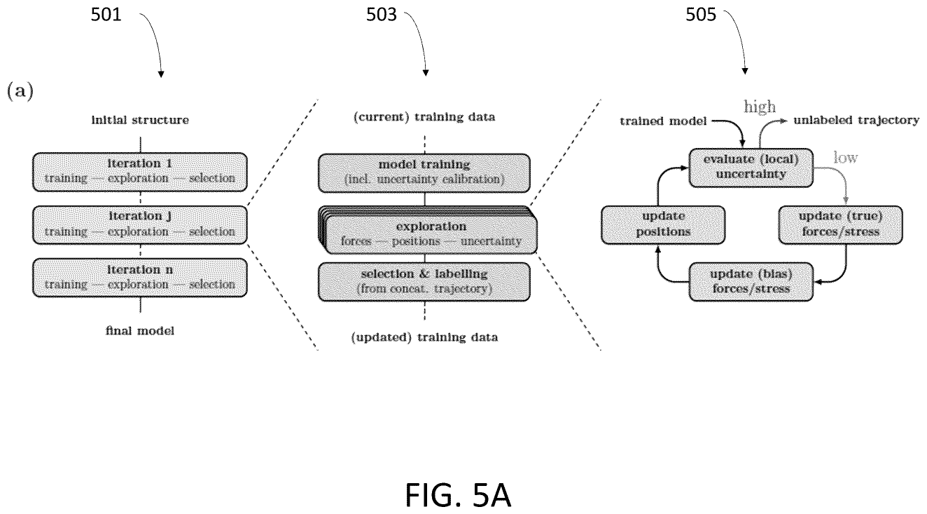

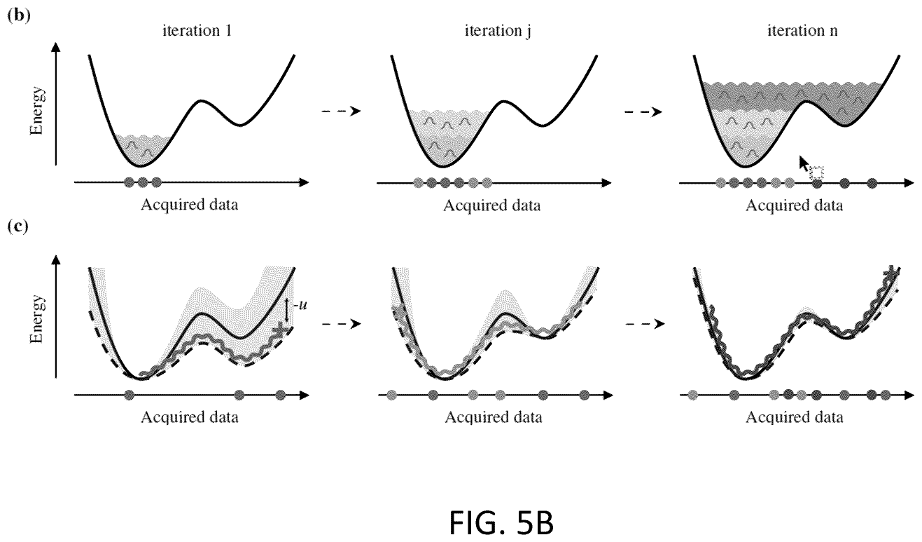

In an embodiment of the present invention, a novel approach to enhance the exploration of the relevant configurational space is introduced, providing the means of data-driven generation of interatomic potentials uniformly accurate across the entire phase space of interest. The embodiment utilizes uncertainty-driven MD simulations—a technique to augment AL pipelines—to explore the entire configurational space of the system. FIG. 5, section (a) presents an overview of the UDAL approach. Adding uncertainty bias to the potential energy is reminiscent of the metadynamics technique, yet it can eliminate the requirement for defining CVs. Uncertainty-driven dynamics can effectively explore configurational spaces, on par with conventional enhanced sampling techniques. A comparison of uncertainty-driven dynamics and metadynamics techniques is depicted in FIG. 5B, in sections (b) and (c). Both approaches change the distribution from which they sample atomic configurations. Uncertainty-driven dynamics aims for reducing the uncertainty associated with the true error, while metadynamics uniformly explores the space of CVs. So, the latter does not ensure uniform accuracy across the relevant configurational space.

FIGS. 5A and 5B provide an overview of UDAL in comparison to its conventional enhanced sampling counterpart, metadynamics. In section (a) of FIG. 5A, an UDAL experiment is initialized by training a first-generation MLIP on a small set of randomly perturbed initial configurations. As shown in graph 501, in each iteration, the current MLIP is utilized to perform multiple MD simulations in parallel, biased towards unexplored regions. Each simulation runs until a threshold criterion, such as a total or atom-based uncertainty threshold value, is met. A batch of configurations is simultaneously chosen from the concatenated trajectory. As shown in graph 503, the selected samples are subsequently fed into a DFT solver, generating energies and atomic forces that update the training data set. As shown in graph 505, this updated dataset is then used to train the next-generation MLIP. With respect to section (b) of FIG. 5B, in metadynamics, exploration of the CV space is achieved by adding Gaussian functions to a history-dependent bias. Long simulation times might be necessary until the phase space of interest has been covered due to the diffusive motion along CVs. With respect to section (c) of FIG. 5B, uncertainty-driven dynamics aims to reduce uncertainty associated with true error, thereby facilitating faster exploration of the phase space of interest. Unlike metadynamics, it can operate without the need for defining CVs.

Ensembles of MLIPs can be used to perform uncertainty quantification and for UDAL. Current approaches that try to avoid ensembles mainly involve computing distances between last layer features or fitting Gaussian mixture models on them. An embodiment of the present invention can use (total) ensemble-free uncertainties derived from sketched gradient features. An atom-based version of ensemble-free uncertainties can be developed and, following calibration with CP, employed as a sensitive metric of local structural changes within the system during dynamics. CP is used for re-scaling atom-based uncertainties from arbitrary units and aligning the highest atomic force error with an equivalent uncertainty value. Having a sensitive metric to monitor local conformational changes can be crucial in some situation, as it can help prevent MD simulations from entering highly extrapolative regions and triggers first-principle calculations only for meaningful atomic configurations.

An embodiment of the present invention can combine (total) ensemble-free uncertainties with AD to perform uncertainty-driven MD simulations, extending beyond the application of AD in adversarial training by dynamically generating an unlabeled data pool through MD simulations. To enhance accessible configurational spaces through uncertainty-driven dynamics, a novel approach for exploring pressure-induced phase transitions in material systems in introduced, by computing uncertainty gradient with respect to strain deformations. To further enhance efficiency of UDAL multiple parallel MD simulations can be utilized in combination with batch selection algorithms, which choose multiple data points at once, to ensure the generation of a highly diverse training data set.

The performance of the embodiment can be assessed based on at least two benchmark systems: alanine dipeptide and MIL-53(Al). Alanine dipeptide is a widely used model for protein back bone structure which allows for the systematic evaluation of the enhanced exploration of configurational spaces by uncertainty driven dynamics. To measure the coverage of the respective CV space, a tree-based weighted recursive space partitioning is introduced. The efficiency of phase space exploration is evaluated by analyzing root-mean-square errors (RMSEs) in energies and atomic forces, and comparing the results to those obtained with high-temperature MD simulations. Furthermore, the rates at which CV space of alanine dipeptide is covered is investigated. MIL-53(Al) is a flexible metal-organic framework with two distinct stable states: closed- and large-pore configurations, which is employed to investigate the enhanced sampling of its cell parameters and contrast the obtained results to the well-established techniques such as metadynamics and high-pressure MD simulations. Finally, the extent to which UDAL results in uniformly accurate potentials is assessed within the phase spaces of interest, and the rates at which the corresponding space can be explored is investigated.

Results: Ensemble-free uncertainties. An atomic configuration is defined,

$$S = {\{ {r}_{i} , {Z}_{i} \}}_{i = 1}^{{N}_{a t}} ,$$

where rᵢ∈ [symbol] ³ are Cartesian coordinates and Zᵢ∈ [symbol] is the atomic number of atom i, with a total of Nₐₜ atoms. The interatomic NN potentials map an atomic configuration to a scalar energy E. The mapping is denoted as f_{θ}: S→E∈ [symbol] , where θ denotes the trainable parameters. Assuming the locality of interatomic interactions, the total energy of the system decomposes into individual atomic contributions, as follows:

$$E ( S , θ ) = {\sum }_{i = 1}^{{N}_{a t}} {E}_{i} ( {S}_{i} , θ ) \qquad ( 1 )$$

where Sᵢ is the local environment of atom i, defined by the cut off radius r_{c}. The Gaussian moment neural network (GM-NN) approach for modeling interatomic interactions is employed.

Different from conventional approaches, ensemble-free uncertainties are acquired by expanding the energy expression around the locally optimal parameters θ* of a trained model, as follows:

$$E ( S , θ ) \approx E ( S , {θ}^{*} ) + {( θ - {θ}^{*} )}^{T} {∇}_{θ} E ( S , θ ) {\vert}_{θ = θ} * \qquad ( 2 )$$

The (total) uncertainty is interpreted as a linear response of a model to small perturbations, defined by the gradient features, φ(S)=∇_{θ}E(S, θ)|_{θ=θ*}. Additionally, due to the locality assumption, gradient features can be expressed as the sum of their atomic contributions:

$$ϕ ( S ) = {\sum }_{i = 1}^{{N}_{a t}} ϕ ( {S}_{i} ) .$$

This realization further motivates the approach of defining atom-based ensemble-free uncertainties.

Ensemble-free uncertainties can be defined by employing squared distances between gradient features or alternatively their posterior transformation (treating Eq. (2) as Bayesian linear regression),

$${u}^{2} ( {S}_{( i )} ) = {u}_{d i s t}^{2} ( {S}_{( i )} ) = {min}_{{s}_{( i )}^{′} ∈ {X}_{train}} {‖ ϕ ( {S}_{( i )} ) - ϕ ( {S}_{( i )}^{′} ) ‖}_{2}^{2} \qquad ( 3 )$$

$$and$$

$${u}^{2} ( {S}_{( i )} ) = {u}_{p o s t}^{2} ( {S}_{( i )} ) = {λ}^{2} {ϕ ( {S}_{( i )} )}^{T} {( {ϕ ( {X}_{t r a i n} )}^{T} ϕ ( {X}_{train} ) + {λ}^{2} I )}^{- 1} ϕ ( {S}_{( i )} ) \qquad ( 4 )$$

respectively.

They are referred to as the distance- and posterior-based uncertainties. Here,

$${X}_{t r a i n} = {\{ {S}^{( k )} \}}_{k = 1}^{{N}_{t r a i n}}$$

denotes atomic structures contained in the training data set, while λ is the regularization strength. Expressions for both total and atom-based uncertainties remain the same, differing only in the use of total or atom-based features: φ(S) or φ(Sᵢ), respectively. The corresponding kernels, φ(Xₜᵣₐᵢₙ)^{T}φ(Xₜᵣₐᵢₙ), are constructed using either total or atomic gradient features. The total energy and uncertainty are extensive properties that scale with the system's size. Therefore, employing them as defined in Eq. (3) and Eq. (4) could favor the selection of larger structures. To make them intensive, the total gradient features are normalized by the number of atoms, φ(S)→φ(S)/Nₐₜ. Different uncertainties may require distinct selection algorithms that improve their performance in a batch setting. Calculating uncertainties using gradient features can become computationally challenging as it scales quadratically with the size of θ. Therefore, two approximations are introduced: one involving last-layer features and the other utilizing sketched gradient features. The latter, demonstrating stronger correlation with RMS errors, is used in this embodiment. Furthermore, atom-based uncertainties, defined by the distances between gradient features, can require an additional approximation to ensure computational efficiency. For this purpose, a batch selection algorithm is incorporated that maximizes distances within the training set. This allows the identification of the most representative subset of atomic features, reducing the computational cost during uncertainty calculations.

The derived total and atom-based uncertainties are initially presented in arbitrary units, which introduces a challenge when integrating them into an AL framework. Specifically, it complicates defining appropriate uncertainty threshold values for initiating DFT calculations. Therefore, the integration of conformal prediction (CP) is proposed to re-scale and calibrate ensemble-free uncertainties. CP helps keep the predicted energy or atomic forces within the bounds defined by the calibrated uncertainty. This improves alignment between the largest prediction error and the uncertainty of the corresponding prediction. The only hyperparameter a determines the confidence level of this alignment.

CP comprises the following steps: (1) A subset of calibration data, sized N_{calibr}, is selected, and the corresponding errors are computed on this subset. For atomic forces, RMSE values

$$Δ {F}_{i}^{2} = {‖ {F}_{i} - {F}_{i}^{r e f} ‖}_{2}^{2} / 3$$

are employed, while for total energies the respective energy absolute errors per atom, Δe=|E−E^{ref}|/Nₐₜ, are used. (2) The uncertainty u(S) is calculated for this subset of data. (3) The ratio Δe/u(S) or ΔFᵢ/u(Sᵢ) is computed. (4) Utilizing quantile regression, the (N_{calibr}+1)(1−α)/N_{calibr}-th quantile, denoted as s, is determined. (5) This s value is then applied to new observations, resulting in the re-scaled and calibrated uncertainty, u→s·u.

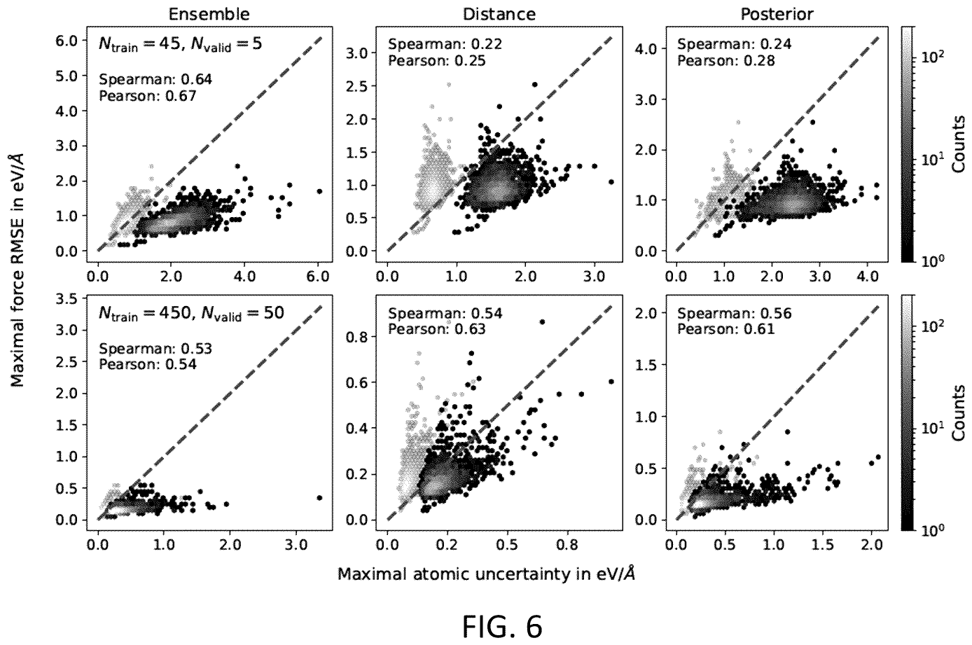

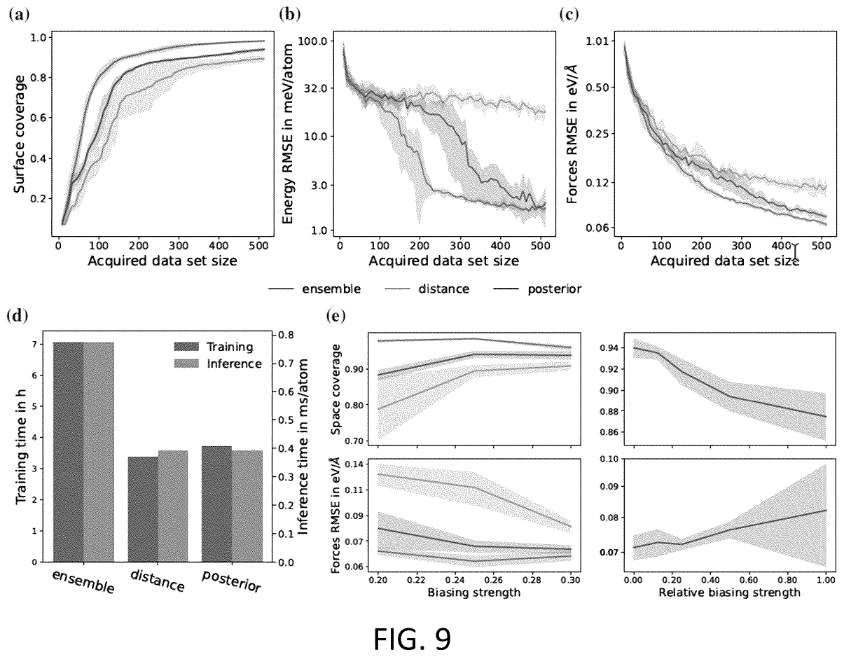

FIG. 6 illustrates the correlation between the derived atom-based uncertainties and the actual RMSEs in atomic forces for the MIL-53(Al) system, evaluated on the test data set. Furthermore, the proposed ensemble-based uncertainty qualification technique is compared. For the latter, the uncertainty of atomic forces is defined as:

$${u}^{2} ( {S}_{i} ) = {u}_{e n s}^{2} ( {S}_{i} ) = \frac{1}{3 M} {\sum }_{j = 1}^{M} {‖ {F}_{i , j} - {\overline{F}}_{i} ‖}_{2}^{2} \qquad ( 5 )$$

where M is the number of model in the ensemble and is set to three in all experiments. The experiments demonstrated that M=3 is sufficient to obtain good performance. Using larger ensembles could make the ensemble approach less advantageous compared to the discussed embodiment; see FIG. 9, section (d) for a comparison of training and inference times. This is because the associated computational costs could rise significantly without providing substantial performance gains.

All models in FIG. 6 are trained using either 45 or 450 configurations from the original training data set, while 5 or 50 configurations are reserved for validation and calibration, respectively. Configurations were selected that were generated using an incremental learning approach. Incremental learning is an iterative learning algorithm which improves MLIPs by training on configurations generated sequentially over time. Thus, these results correspond to the first and tenth iterations in their process. Beyond the context of limited data availability, the results achieved through training and calibration on 45 and 5 configurations, respectively, shed light on the performance of the presented approaches when applied to randomly perturbed configurations of the initial setup.

As shown in FIG. 6, ensemble-free uncertainties show a weaker correlation compared to the ensemble-based approach, when trained and calibrated on 45 and 5 randomly distorted structures, respectively. While ensemble-free embodiments might attribute higher uncertainty values to configurations with high true errors, they tend to overestimate the uncertainty of configurations with a smaller error compared to ensemble embodiments. This leads to a denser range of uncertainties and, thus, worse correlation coefficient. Increasing the sizes of the training and calibration data sets improves the correlation of ensemble-free uncertainties with the true force RMSEs. Ensemble-free approaches were observed to slightly outperform the ensemble-based counterpart. In contrast to the results obtained for MIL-53(Al), ensemble-free embodiments consistently outperform the ensemble-based embodiments on the alanine dipeptide data set. FIG. 6 illustrates that CP helps align the largest force RMSE with the corresponding uncertainty values, which advantageously provides for terminating atomistic simulations before exploring unphysical configurations.

In the context of AL, where an unlabeled pool of data is generated dynamically, it can be crucial in some situations that the attributed uncertainty effectively indicates the onset of extrapolative regions. This possibly essential property remains consistent across all the employed methods, irrespective of whether the models are trained on randomly perturbed configurations or not. Making use of CP allows for the improvement of the respective property even further as demonstrated in FIG. 6. Transparent hexbin points correspond to a confidence level of α=0.5, while opaque points represent α=0.05. CP shifts the hexbin points to be on or below the diagonal line. The latter shows the ideal correlation. Thus, CP helps align the highest error in atomic forces with an equivalent uncertainty value. Calibration can also be crucial in some settings for ensemble-based uncertainties, owing to possible deviations from ideal Gaussian behavior in the underlying distribution. Overall, ensemble-free uncertainties were observed to exhibit performance on par with their ensemble counterparts while providing the advantage of reduced computational costs compared to the latter.

UDAL: In the following, the presented uncertainty estimates are combined with AD, different from methods that rely on analytic derivatives. While AD in the context of constructing training data sets was previously used solely for adversarial training, its application to a setting involving the dynamic generation of an unlabeled data pool is unexplored. Furthermore, using AD in the context of UDAL enables going beyond MD driven by the bias forces and facilitates the exploration of new configurational and chemical spaces.

The biased energy is expressed as:

$${E}^{b i a s e d} ( S , θ ) = E ( S , θ ) - τ u ( S , θ ) \qquad ( 6 )$$

The negative sign drives the system toward uncertain regions. Utilizing the bias forces acting on atom i, denoted as −∇ᵣ_{ᵢ}u(S,θ), formulating the total biased force on atom i is as follows:

$${F}_{i}^{b i a s e d} ( S , θ ) = {F}_{i} ( S , θ ) + τ {∇}_{{r}_{i}} u ( S , j ) \qquad ( 7 )$$

While this approach can be used to bias configurational space exploration, certain periodic systems can require enhanced sampling of their cell parameters. For this purpose, the concept of bias stress is introduced, defined by ∇_{ε}u(S,θ,ε)|_{ε=0}/V with V denoting the volume of the periodic cell. The expression for the total biased stress is as follows:

$${σ}^{b i a s e d} ( S , θ ) = σ ( S , θ ) - τ \frac{1}{V} {∇}_{ε} u ( S , θ , ε ) {\vert}_{ε = 0} \qquad ( 8 )$$

Here, u(S,θ,ε) denotes the uncertainty after a strain deformation with symmetric tensor ε∈ [symbol] ^{3×3}, i.e., r→(1+ε)·r. This bias stress alters the internal pressure of the atomic system and is used in MD simulations run under constant pressure conditions (NpT statistical ensemble). In other words, the incorporation of the bias term reduces internal pressure, thereby allowing for the exploration of pressure-induced transitions in bulk materials.

Gradients of uncertainties with respect to atomic positions or strain deformations might exhibit different magnitudes compared to the corresponding gradients of total energy. Hence, to ensure a consistent driving toward unexplored regions, a re-scaling of the respective values may be necessary. A re-scaling strategy is proposed that tracks the magnitudes of true and biased atomic forces and stresses over a duration of N steps, subsequently computing the ratio between them. For atomic forces, the re-scaling can be achieved through the following expression:

$$τ ( t ) = τ ( t = 0 ) \times \frac{\sqrt{{\sum }_{n = 1}^{N} {‖ {F}_{t - n Δ t} ‖}_{2}^{2}}}{\sqrt{{\sum }_{n = 1}^{N} {‖ {∇}_{{r}_{i}} {u}_{t - n Δ t} ‖}_{2}^{2}}} . \qquad ( 9 )$$

An equivalent expression holds for re-scaling the bias stress. The rescaling of gradients, as demonstrated above, bears similarity to the AdaGrad algorithm, which dynamically adjusts the learning rate (analogous to the biasing strength in this context) based on historical gradients from previous iterations. Although incorporating momentum i.e., using exponential moving averages, could potentially improve the AdaGrad approach, treating all past gradients with equal weight seems essential within the context of the embodiment, which is based on MD simulations. Attempts to damp learning along directions with high curvature, like the oscillatory modes addressed by the Adam optimizer did not yield improved performance for the embodiment. Employing species-dependent biasing strengths in biased atomic forces, τ→τ_{Z}_{ᵢ}, with a particular emphasis on damping hydrogen biasing, can improve the efficiency of enhanced sampling in UDAL.

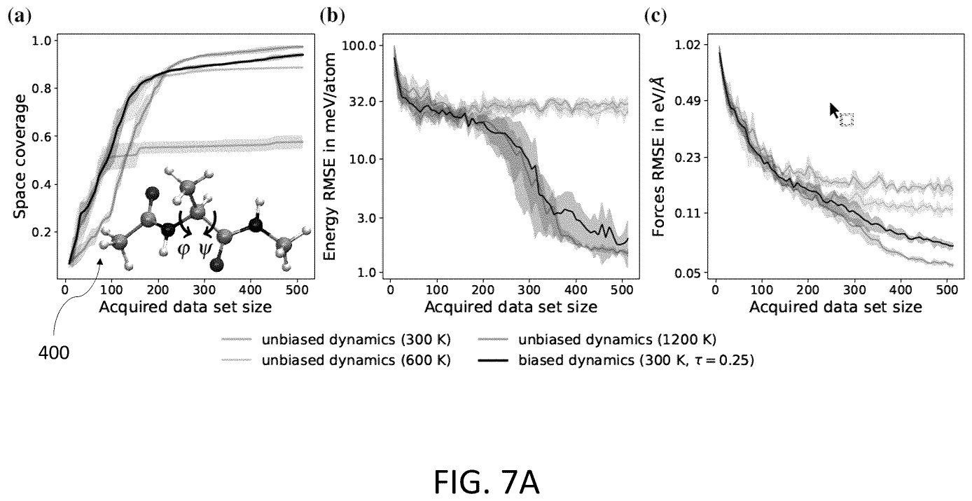

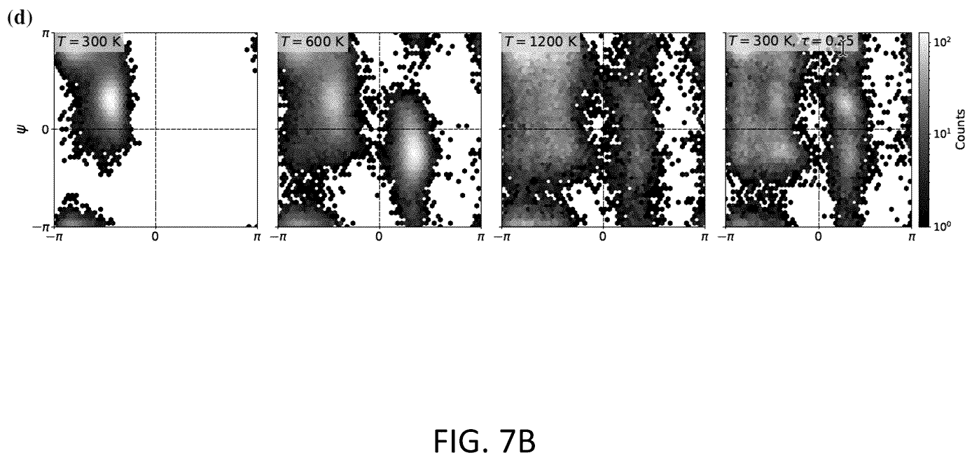

Collective variable sampling in alanine dipeptide. The UDAL approach can be applied to develop an MLIP for alanine dipeptide and investigate enhanced sampling of its collective variables using uncertainty-driven MD. Additionally, the results of uncertainty-driven approach can be compared to those of naive AL. To manage computational demand, the recent AMBER ff19SB force field for reference energy and atomic force evaluations (see FIGS. 5A and 5B) can be utilized, for which a PyTorch implementation by TorchMD was used. The test data set comprised 2000 configurations drawn randomly from a MD trajectory, generated by running MD simulation over 1 ns with a time step of 0.5 fs in the NVT statistical ensemble at a temperature of T=1200 K. A Langevin thermostat was employed with a friction coefficient of 0.02 in atomic units as implemented within the ASE simulation package. This data set covers the relevant phase space of alanine dipeptide and provides an upper limit to sampling collective variables (CVs) of it. Each AL experiment began by randomly perturbing the initial alanine dipeptide configuration. The latter has been obtained by performing geometry optimization with the BFGS algorithm. The first-generation MLIP, trained to this initial data set, was then used to run MD simulations. Eight parallel MD simulations were employed, each running for a maximum of 10 ps with a time step of 0.5 fs. The maximal allowed data set size was set to configurations, which results in a maximum of 64 AL iterations. An uncertainty threshold value of 1.5 eV/Å was employed, defined empirically. A lower threshold value results in slower CV space exploration, while a larger one leads to the exploration of distorted, unphysical configurations. A force threshold of 20 eV/Å was used for reference calculations to avoid training with strongly distorted structures, although these were observed primarily at very high biasing strength values (a smaller integration time step might be used equivalently). Unbiased, naive AL experiments were carried out at three distinct temperatures: 300, 600, and 1200 K. The UDAL experiments were run at 300 K. The high-temperature experiments at 1200 K could be seen as the upper limit for learning the system's behavior across its entire phase space. All experiments had been repeated five times. FIGS. 7A and 7B illustrate the performance of the models obtained for alanine dipeptide depending on the number of acquired data points. The presented results are provided for the posterior-based uncertainty, as defined in Eq. (4). In FIG. 7A, section (a), the CV space coverage is presented defined by the two dihedral angles (φ and ψ, see inset 701 of FIG. 7A) obtained depending on the number of acquired data. To measure the coverage of the CV space, a tree-based weighted recursive space partitioning is employed. Naive AL experiments at 1200 K achieve the highest coverage of 0.97±0.01, surpassing 0.58±0.03 and 0.89±0.01 coverages achieved by their 300 K and 600 K counterparts. However, the CV space is covered by high-temperature experiments comparatively slow until the data set accumulates about 100 configurations, in contrast to other temperatures. This observation can be attributed to stronger local perturbations of the systems, leading to a faster onset of extrapolation before a larger CV space could be explored. Running preliminary UDAL experiments resulted in CV 380 space coverages of about 0.87±0.02, close to those obtained at 600 K. It was found that the uncertainty-driven sampling of CVs is mainly hindered by fast moving hydrogen atoms. Species-dependent biasing strengths appeared to improve the sampling efficiency and resulted in CV space coverages of 0.94±0.01 for the biasing strength of hydrogen of 0.0, as shown in FIG. 7A, section (a). Furthermore, uncertainty-driven experiments were observed to surpass their high-temperature counterparts in the data-scarce regime, resulting in a steeper slope and, thus, faster exploration of CV space up to approximately 100 acquired data points. These findings demonstrate that UDAL facilitates the efficient sampling of CVs in alanine dipeptide at 300 K, in comparison to temperature-driven naive AL experiments, in both data-scarce and data-rich regimes.

FIG. 7A, sections (b) and (c), depicts learning curves for total energies and atomic forces, respectively, as a function of the acquired data. Both learning curves start to improve upon the 300 K experiments after acquiring about 100 configurations, which aligns with covering larger CV space as shown in FIG. 7A, section (a). High-temperature and uncertainty-driven experiments yield 400 similar RMSE values in energies, resulting in 1.5 and 2.0 me V/atom, respectively. In contrast, their 300 and 600 K counterparts exhibit values larger than the aforementioned 403 RMSEs by a factor of more than 13. Similar trend is observed for RMSE in atomic forces. The high-temperature and uncertainty-driven experiments yield RMSE values in atomic forces of 0.055 and 0.071 eV/Å, respectively. In comparison, their 300 and 600 K counterparts yield corresponding RMSE values of 0.149 and 0.116, respectively. FIG. 7A illustrates that AL experiments with uncertainty-biased MD simulations at 300 K outperform their unbiased counterparts at 300 K and 600 K and achieve comparable performance to those run at 1200 K.

Ramachandran plots in FIG. 7B, section (d), provide a visual representation of the sampled CV space for the four investigated experimental settings. The uncertainty-driven experiments, performed at 300 K, sampled CV space comparable to high-temperature naive AL experiments. AL experiments performed at 600 K manage to explore the second basin, which is expected based on the experimental setup. However, it is not able to explore other high-energy transitions as efficiently as 1200 K and uncertainty-driven experiments. AL experiments performed at 300 K explore only the initial basin. These results serve as a strong evidence of rare event exploration through uncertainty-driven dynamics, not provided before. Uncertainty-driven approaches were observed to manage to sample two slow modes in alanine dipeptide, despite that 27 degrees of freedom (heavy atoms) were effectively biased, proving its efficiency. FIG. 7B illustrates that uncertainty-biased MD simulations at 300 K explore both stable states of alanine dipeptide (that are not explored by unbiased simulations at 300 K) and reach a comparable CV space coverage to simulations at 1200 K.

FIGS. 7A and 7B provide a comparison of naive and uncertainty-driven AL approaches for the alanine dipeptide molecule. All methods use ensemble-free posterior-based uncertainties. Unlike the naive AL method, which relies solely on ensemble-free uncertainties to terminate MD simulations, UDAL utilizes these uncertainties for both exploration and stopping criteria. The analysis is based on three metrics: graph (a) illustrates the coverage of the CV space; graph (b) illustrates the RMSEs in predicted energies; and graph (c) illustrates the RMSEs in atomic forces. The investigated system, alanine dipeptide, is depicted as an inset 701 in section (a), including its CVs, φ and ψ. FIG. 7B, section (d) provides Ramachandran plots illustrating the CV spaces explored by the four experimental setups.

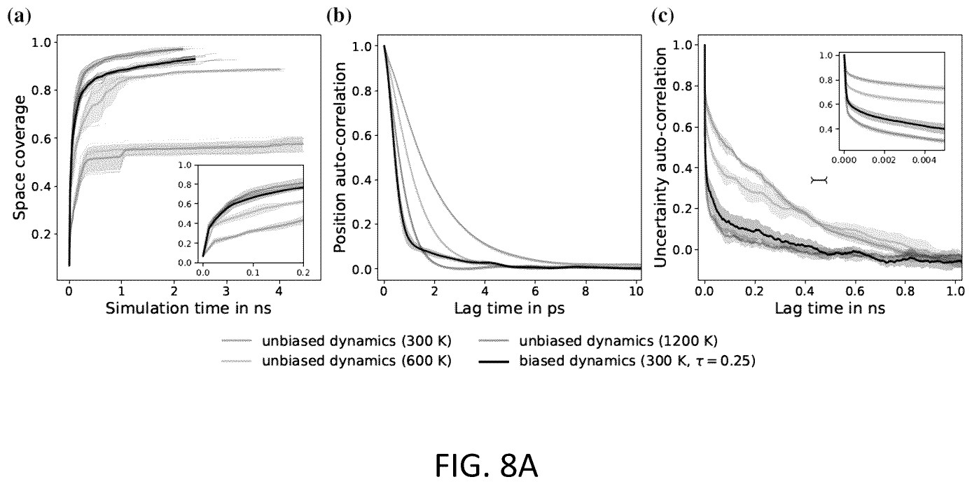

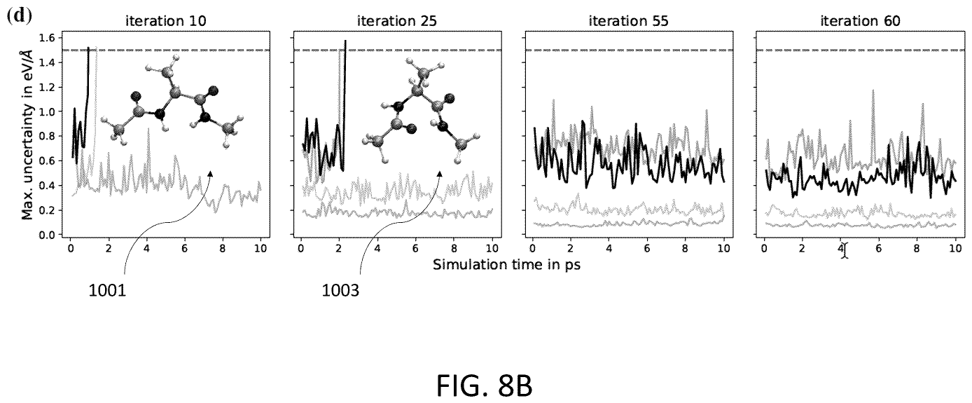

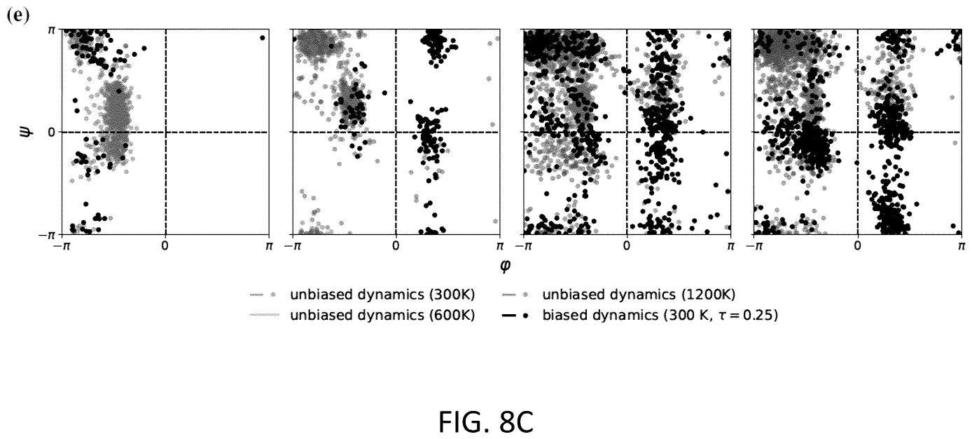

FIGS. 8A and 8B investigate the enhanced sampling of alanine dipeptide's phase space in more details. From FIG. 8A, section (a), where the CV space coverage is plotted as a function of simulation time, uncertainty-driven and high temperature naive AL experiments were observed to require shorter MD simulations to achieve the performance of experiments run at 300 and 600 K. The coverage the 300 K counterparts (0.58±0.03) is already reached after 55 and 60 ps of high-temperature and uncertainty-driven simulation, respectively. To reach the coverage of the 600 K naive AL experiments (0.89±0.01), high-temperature and uncertainty driven experiments required 0.4 and 1.2 ns, respectively. The 300 and 600 K naive AL experiments reach their final coverages after running MD simulations for about 4 ns. From the inset 801 of FIG. 8A, section (a), high-temperature and uncertainty-driven experiments are observed to have same initial slopes, indicating comparable sampling efficiency.

FIG. 8A, sections (b) and (c) show auto-correlation functions of positions and atom-based uncertainties, providing insights into the rates at which new atomic configurations in the positions and uncertainty spaces are explored. A faster decay of an auto-correlation function is associated with a faster exploration of the respective space. Position and uncertainty auto-correlation functions exhibit similar behavior in uncertainty-driven and high-temperature naive AL experiments. They both indicate faster exploration of positions and uncertainty spaces compared to their 300 and 600 K counterparts. Position auto-correlation times of 0.74 and 0.69 ps were found for high-temperature and uncertainty-driven experiments, respectively.

The respective values for uncertainty auto-correlation functions are 21.3 and 52.8 ps. For the 300 K experiments auto-correlation times larger by a factor of 2.8-3.0 and 6.2-15.3 were found for positions and atom-based uncertainties, respectively. The respective factors for the 600 K experiments are 1.7-1.8 and 4.9-12.0. FIG. 8A illustrates that uncertainty-biased simulations at 300 K explore the configurational space of alanine dipeptide at rates comparable to simulations at 1200 K, outperforming their counterparts at 300 K and 600 K. The exploration rate is analyzed using auto-correlation functions, for which faster decay indicates faster exploration of the respective space (position and uncertainty spaces are investigated).

FIGS. 8A, 8B and 8C provide a comparison of the efficiency of sampling between naive and uncertainty-driven AL approaches for the alanine dipeptide molecule. All methods use ensemble-free posterior-based uncertainties. Unlike the naive AL method, which relies solely on ensemble-free uncertainties to terminate MD simulations, UDAL utilizes these uncertainties for both exploration and stopping criteria. The analysis is based on three metrics: section (a) illustrates the coverage of the CV space; section (b) illustrates the auto-correlation functions of atomic positions; and section (c) illustrates the auto-correlation functions of atom-based uncertainties. Section (d) illustrates the time evolution of the maximal atom-based uncertainty within an AL iteration and the entire experiment. Time evolution is shown for one of the eight MD simulations. The dashed gray line represents the uncertainty threshold value of 1.5 eV/Å. For the uncertainty-driven experiment, configurations which reached the uncertainty threshold are shown in insets 1001, 1003. Section (e) illustrate Ramachandran plots illustrating the exploration of CV space over AL iterations presented in section (d). Simulation time refers to the effective number of MD steps (×0.5 fs) required to reach the final coverage, while lag time denotes the time interval between two successive MD frames.

FIGS. 8B and 8C, sections (d) and (e) show the time evolution of the maximal atom-based uncertainty and the sampled CV space within an AL iteration. The exploration of CVs by the 300 K experiment is restricted to the initial basin, leading to the overall reduced uncertainty level across the experiment compared to high-temperature and uncertainty-driven settings. Increasing the temperature in naive AL or switching on uncertainty driving results in faster exploration of high-uncertainty regions and, thus, shorter MD trajectories. The uncertainty level of 1200 K and uncertainty-driven experiments remain relatively high compared to the 600 K counterpart, which aligns well with the efficient sampling of the CV space also at later iterations. These findings suggest that uncertainty-driving leads to interatomic potential models which are uniformly accurate across the relevant phase space. FIG. 8B illustrates time evolution of maximal atom-based uncertainty for unbiased and uncertainty-biased MD simulations. Uncertainty-biased simulations advantageously explore, on average, regions with higher uncertainty levels. FIG. 8C illustrates that uncertainty-biased MD simulations advantageously simultaneously explore regions with higher uncertainty levels and rare events (transitions that otherwise require longer simulation time).

FIG. 9, sections (a)-(c) present the comparison of ensemble-free and ensemble-based AL approaches. The same metrics are employed as in FIG. 9, sections (a)-(c). The posterior-based approach almost reaches the performance of the ensemble-based counterpart in energy and atomic forces RMSEs, differing only by a factor of 1.1. The ensemble-based embodiment reaches also an coverage of CV space larger by 0.04 compared to the posterior based experiments. The latter improvement is due to running MD using an ensemble, leading to larger slopes at the onset of the experiment. The improvement in RMSEs can be attributed to the statistical fluctuations. Distance-based experiments appear to achieve the worst performance compared to ensemble- and posterior based approaches.

Comparing training and inference times in FIG. 9, section (d), ensemble-free approaches were observed that are at least two times more efficient compared to ensemble-based approaches. Parallelized training and inference of ensembles were used for this comparison. Based on the results from FIG. 7(a)-(c), using larger ensembles was expected to improve their performance marginally, while rising the computational cost significantly. FIG. 9, section (e), compares the performance trade-offs of presented AL approaches with the biasing strength, absolute and the hydrogen's relative one. Previously, the latter significantly impacted the results. The performance of the distance-based approach was observed to depend more on the biasing strength value compared to other methods. While ensemble-free approaches stay constant or continue improving with larger values of biasing strength, the performance of the ensemble-based approach deteriorates after reaching a biasing strength of 0.25. Employing the posterior-based approach, it was observed that changing hydrogen's relative biasing strength from 1.0 to 0.0 improves its performance by a factor of 1.08 and 1.15 for coverages and atomic forces RMSEs, respectively. This finding opens a question on the data-driven re-distribution of biasing strengths to allow the optimal uncertainty-driven exploration.

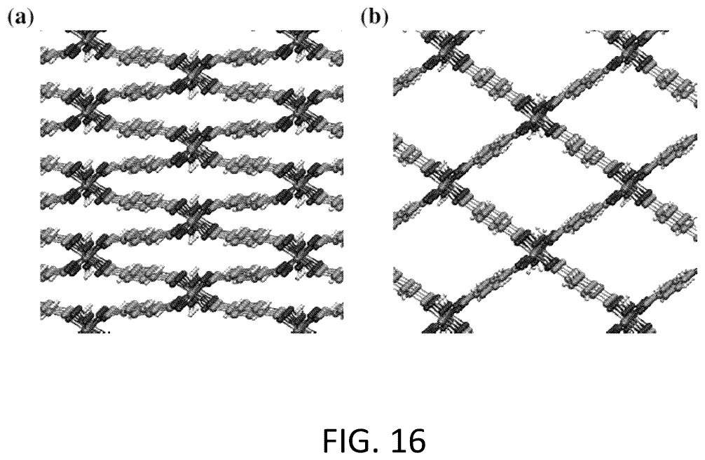

Sampling cell parameters in MIL-53(Al). The UDAL approach was applied to develop an MLIP for MIL-514 53 (Al), a flexible metal-organic framework featuring distinct 515 closed-pore (left) and large-pore (right) stable phases, see FIG. 16. Driving an MD simulation at constant pressure (NpT statistical ensemble) toward the transition between both stable phases was investigated, using solely the stress bias as presented in Eq. (8).

Sampling the entire phase space of MIL-53(Al) is essential in some situations for accurately modeling the system's behavior under external pressure. Different from the traditional metadynamics enhanced sampling approach, which employs cell parameters as CVs, the internal pressure was reduced to facilitate phase transition sampling. Interatomic potentials trained using uncertainty driving were compared to naive AL and incremental learning, which employ high-pressure MD and metadynamics, respectively. The quantum mechanical calculations employ DFT at the PBE-D3(BJ) level.

Each AL experiment began by randomly perturbing the atom positions and the cell parameters of the initial MIL-53(Al) structure in its closed-pore stable phase. The initial data set comprised 32 configurations, 90% or which are used for training and the remaining configurations were reserved for validation and uncertainty calibration. These data were used to train the first-generation MLIP and run 32 parallel MD simulations for a maximum of 10 ps with a time step of 0.5 fs in the NpT statistical ensemble. Particularly, the isobaric-isothermal form of the Nosé-Hoover dynamics was employed, as implemented in ASE. The characteristic time scales of the thermostat and barostat were set to 0.1 and 1 ps, respectively.

The maximal allowed data set size was set to 512 configurations, which resulted in a maximum of 16 AL iterations. An uncertainty threshold value of 1.0 eV/Å was employed. A force threshold of 20 eV/Å is used for reference calculations. All experiments were run at a fixed temperature of 600 K. The external pressure was set to 0 MPa, or +250 MPa, each for the half of 32 parallel MD simulations. All experiments were repeated for three times.

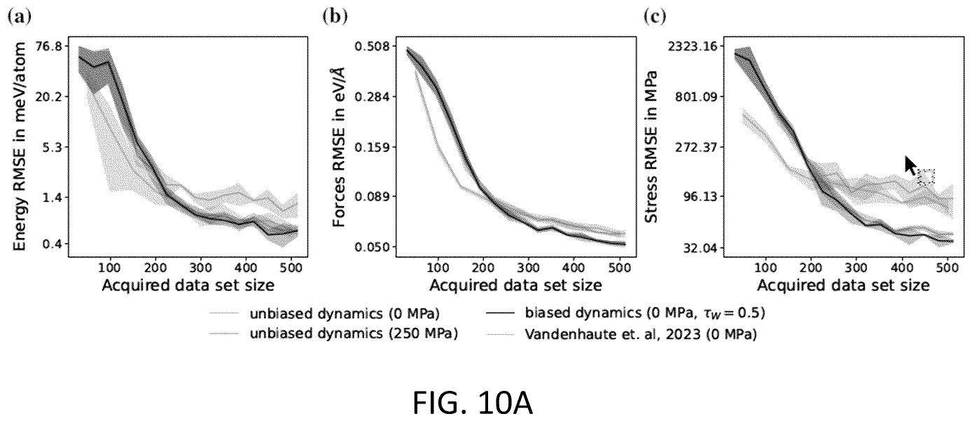

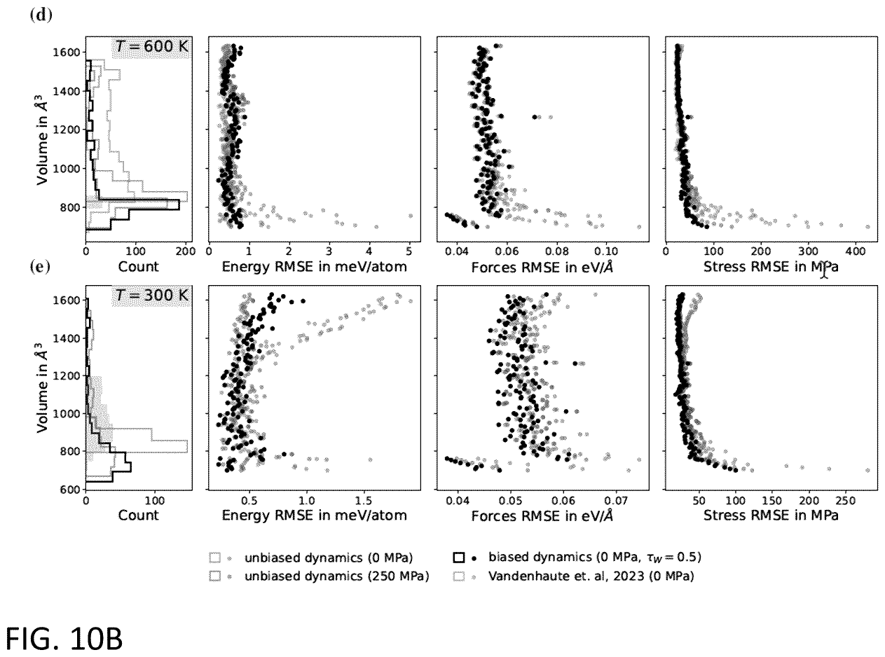

FIG. 10A, sections (a)-(c) compares RMSEs in energies, atomic forces, and stresses obtained for uncertainty-driven experiments to those obtained by running naive AL experiments, or by training an MLIP on the data set obtained in an incremental learning setting combined with metadynamics. The models obtained by training on metadynamics data were observed to slightly outperform the others for small data sets sizes. This might be attributed to different initialization, including the generation of perturbed configurations, and experimental setting of incremental learning compared to AL employed here. Incremental learning does not use the uncertainty thresholds and runs MD simulations for 1 ps each. FIG. 10A illustrates that AL experiments with uncertainty-biased MD simulations at 0 MPa outperform their unbiased counterparts at 0 MPa and 250 MPa. They also outperform experiments based on metadynamics simulations.

After reaching a data set size of about 250 configurations, the uncertainty-driven and high-pressure MD experiments outperform their counterparts based on metadynamics and zero-pressure MD. RMSEs were obtained in total energies of 0.58±0.10, 1.17±0.36, 0.57±0.05, and 0.57±0.08 meV/atom for the experiments based on metadynamics, zero and high-pressure, as well as on uncertainty-driving, respectively. For RMSE values in atomic forces, the high-pressure and uncertainty-driven experiments reach comparable values of 0.052±0.001 and 0.051±0.001 eV/Å, respectively, and outperform the other approaches by a factor of 1.14. Even stronger improvement compared to experiments based on metadynamics and zero-pressure was achieved on stress RM578 SEs. The high-pressure and uncertainty-driven experimental embodiments reached RMSEs of 42.7±1.4 and 36.6±1.5 MPa, outperforming others by a factor of 1.8-2.5.

FIG. 9 compares ensemble-based and ensemble-free uncertainty-driven approaches for the alanine dipeptide molecule, thereby comparing different uncertainty quantification methods. It was found that the ensemble-free gradient-based uncertainties according to embodiments of the present invention often achieve similar or, sometimes, even better performance than ensemble-based approaches, while at the same time considerably reducing the computational cost of the respective simulations. The analysis is based on three metrics: section (a) illustrates the coverage of the CV space; section (b) illustrates the RMSEs in predicted energies; and section (c) illustrates the RMSEs in atomic forces. Individual models were used in the ensemble to obtain respective RMSE values. Section (d) illustrates the comparison of training and inference times required to execute each method. Section (e) illustrates the dependence of CV space coverage and atomic forces RMSEs on absolute and hydrogen's relative biasing strength. For the latter, a posterior-based approach has been used.

FIG. 16 illustrates the structure of the MIL-53(Al) system featuring (a) closed-pore stable phases and (b) large-pore stable phases.

FIGS. 10A and 10B compare naive and uncertainty-driven AL strategies for the MIL-53(Al) system. All methods use ensemble-free posterior-based uncertainties. Unlike the naive AL method, which relies solely on ensemble-free uncertainties to terminate MD simulations, UDAL utilizes these uncertainties for both exploration and stopping criteria. The analysis is based on three metrics: section (a) illustrates the RMSEs in predicted energies; section (b) illustrates the RMSEs in atomic forces; and section (c) illustrates the RMSEs in predicted stresses. Section (d) illustrates the volume distribution for configurations acquired during AL, along with volume-dependent RMSE values for total energies, atomic forces, and stress evaluated on the test data set. Section (e) illustrates the volume distribution for configurations acquired during AL, along with volume-dependent RMSE values for total energies, atomic forces, and stress evaluated on the test data set. These results were obtained for the experiments run at 300 K to reduce the probability of MIL-53(Al) exploring its second stable state. Each experiment had been initialized using 256 configurations, with a volume of less than 1200 Å³, drawn randomly from the original training data.

FIG. 10B, section (d), shows the volume distribution of the configurations acquired during training and the performance of the resulting models on the test set as a function of the volume. Compared to naive zero-pressure experiments, uncertainty driving leads to more uniform sampling of the transition region. The latter approach enhances the exploration of configurations with smaller volumes in comparison to metadynamics and zero-pressure experiments, achieving a similar volume distribution to high-pressure simulations. The training data obtained by incremental learning shows nearly uniform distribution of volumes, which is a typical feature for metadynamics-based approaches. UDAL was observed to outperform other methods as it removes all outliers, except for one in atomic forces. These findings show a primary advantage of the embodiment of the present invention of the uncertainty driven approach compared to metadynamics. The former drives the exploration towards decreasing the error across the entire phase space, while the latter was designed to uniformly explore the phase space. The uniform exploration of a phase space does not necessarily improve the performance of the resulting model on it.

To provide a more solid evidence of enhanced sampling achieved by uncertainty-driving, an experiment was designed which replicated the above experiments, but ran MD simulations at 300 K and was initialized using 256 configurations. These 256 configuration were chosen to have a volume less than 1200 Å³ (closed-pore state) and were drawn randomly from the respective data set. FIG. 10B, section (e), shows that naive AL experiments performed with zero external pressure were not able to leave the closed-pore state of MIL-53(Al). In contrast, uncertainty-driven experiments explore the entire volume space and reach a distribution similar to high-pressure simulations. For RMSE values, zero-pressure experiments were observed to achieve a considerably worse performance for small and larger volumes compared to high pressure and uncertainty-driven AL. Uncertainty-driven experiments show a better performance for smaller volumes compared to high-pressure counterparts, and remove most of the outliers in line with FIG. 10B, section (d). FIG. 10B illustrates that uncertainty-biased MD simulations advantageously cover a larger configurational space compared to their counterparts and achieve uniform distribution of energy, atomic force, and stress RMSEs.

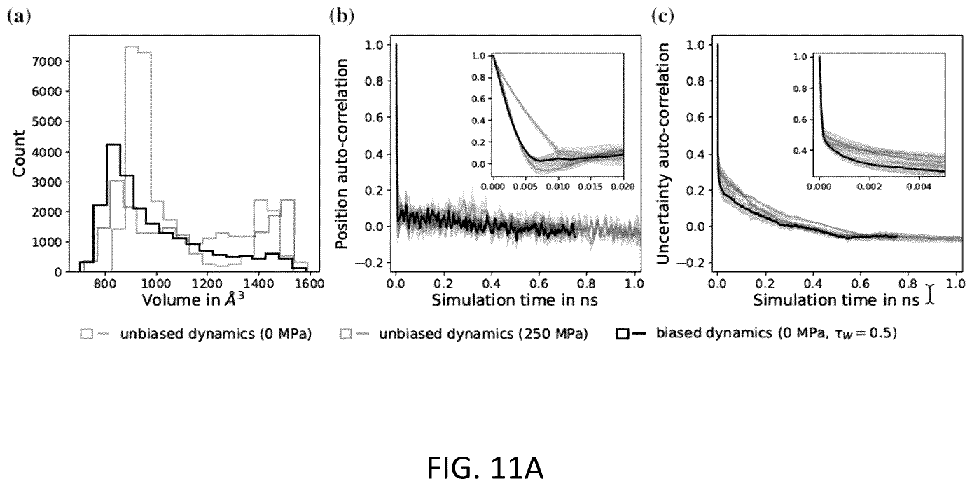

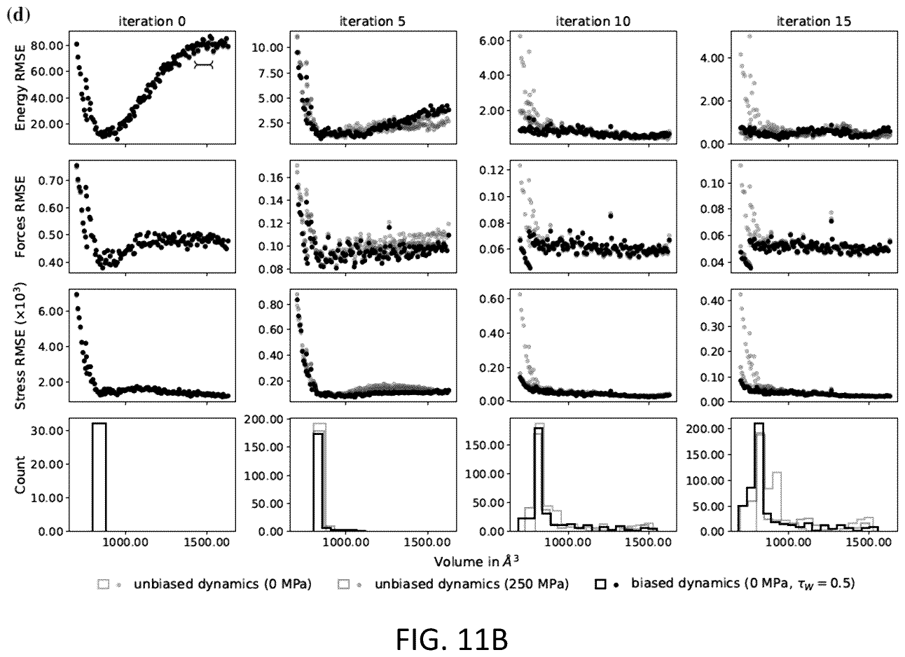

FIGS. 11A and 11B investigates the enhanced exploration of the phase space of MIL-53(Al) in more details. From FIG. 11A, section (a), uncertainty-driven experiments are observed to explore the transition between both stable states more frequently compared to the zero-pressure counterpart, especially considering the counts relative to those observed for the closed-pore state. Furthermore, they target the smaller volumes more frequently compared to the latter, and to the high-pressure experiments, which aligns well with the results obtained in FIG. 11B, section (d). The large pore state is sampled less frequently by the uncertainty-driven experiment compared to the high-pressure one, indicating that it can be accurately modelled trained on configurations from the closed pore state or those which are close to the transition volume. FIG. 11A illustrates that uncertainty-biased MD simulations advantageously explore the position and uncertainty spaces at similar rates to high-pressure simulations without knowing the pressures required to explore the large-pore state of MIL-53(Al). They also drive the dynamics toward exploration of the closed-pore phase for which unbiased and metadynamics-based counterparts fail to make accurate energy, atomic force, and stress predictions. FIG. 11B illustrates that uncertainty-biased MD simulations outperform their counterparts in terms of uniformly distributed RMSEs already during early AL iterations.

FIG. 11A, section (b) and (c), show auto-correlation functions of positions and atom-based uncertainties, similar to the analysis performed for the alanine dipeptide molecule. Position auto-correlation functions decay similarly fast for the high-pressure and uncertainty-driven experiments, while auto-correlation functions for atom-based uncertainties indicate that high-uncertainty regions are reached faster by the latter. Position auto-correlation times were found of 2.08±0.58 and 2.75±0.46 ps for high-pressure and uncertainty-driven experiments, respectively. For the atom-based uncertainty, the respective values of 66.32±2.02 and 44.87±14.08 ps were found. For the zero-pressure experiments, auto-correlation times of 10.60±9.54 and 88.05±2.52 for positions and atom-based uncertainties were found, respectively.

FIG. 11B, section (d), shows the time evolution of the volume distribution of the acquired data and of the RMSE values for energies, atomic forces, and stresses. All experiments were initialized using the same configurations, resulting in the same volume distributions and RMSEs. After five iterations, uncertainty-driven and high-pressure experiments explored larger configurational spaces, resulting in a better performance in RMSEs compared to the zero-pressure counterparts. All RMSE values were similar for both closed and large pore states, except for the configurations with volumes less than 800 Å³. This observation is in line with the fact that uncertainty-driving leads to more efficient exploration of smaller volumes in FIG. 11A, section (a) After ten iterations, uncertainty-driven experiments explored the largest volume space compared to other approaches. Driving the exploration toward smaller volumes resulted in slightly better RMSEs for UDAL compared to high-pressure approach. The last iteration shows an effective driving toward the configurations with smaller volumes, yielding interatomic potentials which are uniformly accurate across the relevant space.

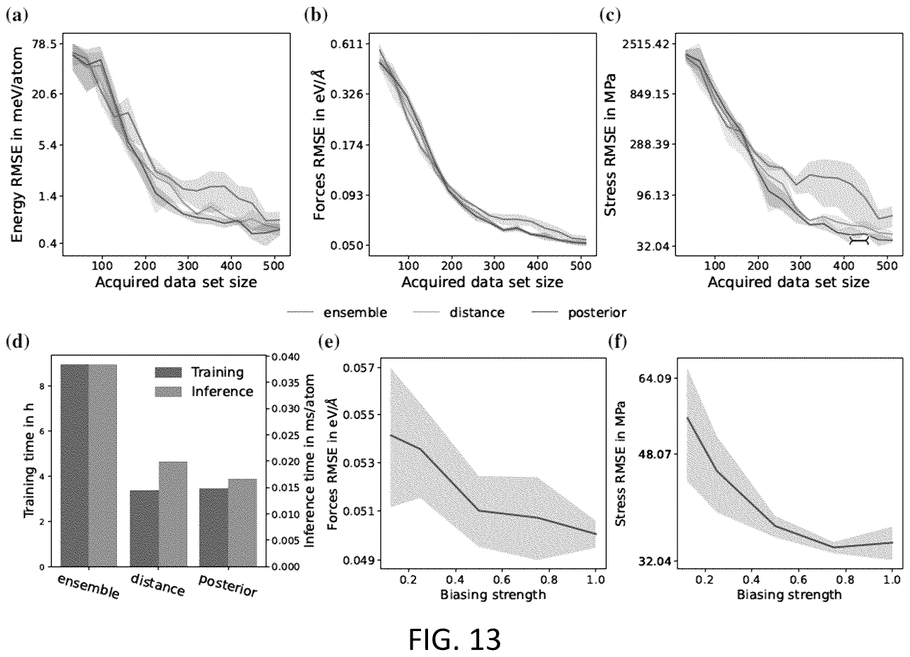

FIG. 13, sections (a)-(c), present a comparison of ensemble-free and ensemble-based AL approaches for MIL-53(Al) as different uncertainty quantification methods, similar to the alanine dipeptide molecule. It was found that the ensemble-free gradient-based uncertainties according to embodiments of the present invention often perform better than ensemble-based approaches, considerably reducing the computational cost of the respective simulations. RMSE values were employed in energies, atomic forces, and stresses to assess the performance of the respective approaches. Different from the results obtained for alanine dipeptide, an ensemble of three models was observed to be continuously outperformed by the ensemble-free approaches. The energy RMSE achieved by ensemble-, distance-, and posterior-based AL are 0.74±0.18, 0.59±0.10, and 0.57±0.08 me V/atom, respectively. For atomic forces, the respective values are 0.054±0.002, 0.051±0.002, and 0.051±0.001 eV/Å. The largest difference in stress RMSEs were found, where posterior-based approach outperforms the ensemble by a factor of 1.7, by reaching RMSE values of 36.6±1.5 and 62.1±13.4 MPa, respectively. The distance based approach achieves an RMSE value of 41.8±3.43 MPa.

FIGS. 11A and 11B compare the sampling efficiency for naive and uncertainty-driven AL approaches for the MIL-53(Al) system. All methods use ensemble-free posterior-based uncertainties. Unlike the naive AL method, which relies solely on ensemble-free uncertainties to terminate MD simulations, UDAL utilizes these uncertainties for both exploration and stopping criteria. The analysis is based on three metrics: section (a) illustrates the volume distribution of configurations sampled over the course of the experiment; section (b) illustrates auto-correlation functions for positions; and section (c) illustrates the auto-correlation functions for atom-based uncertainties. Section (d) illustrates the time evolution of the volume distribution of configurations acquired during training and of RMSE values for total energies, atomic forces, and stresses evaluated on the test data set depending on the unit cell volume.

FIG. 13, sections (a)-(f), compares ensemble-based and ensemble-free uncertainty-driven approaches for the MIL-53(Al) system. The analysis is based on three metrics: graph (a) illustrates RMSEs in predicted energies; graph (b) illustrates RMSEs in atomic forces; and graph (c) illustrates RMSEs in predicted stresses. Individual models were used in the ensemble to obtain respective RMSE values. Graph (d) illustrates a comparison of training and inference times required to execute each method. Graph (e) illustrates a dependence of RMSEs in atomic forces on the stress biasing strength. Graph (f) illustrates a dependence of RMSEs in stresses on the stress biasing strength.

Comparing training and inference times in FIG. 13, section (d), ensemble-free approaches were observed to require about 2.6 and 1.9-2.3 less time compared to ensemble-based approaches, respectively. The posterior-based approach appears to be faster in execution compared to the distance-based one, resulting in 16.7 μs/atoms and 19.9 us/atom, respectively. The respective inference times have been measured for 32 MD simulations run in parallel on a node with 64 AMD EPYC 7543 CPU cores and four NVIDIA A100 GPUs. They include the neighbor list computation. Larger ensembles might improve their performance at the cost of longer training and inference times. FIG. 13, sections (e)-(f), compares the performance of posterior-based UDAL depending on the biasing strength. For the approach of the embodiment which employs stress bias, a continuous improvement was observed of RMSEs in atomic forces and stresses. For atomic forces, RMSE values of 0.054±0.003 and 0.050±0.000 eV/Å for a stress biasing strength of 0.125 and 1.0, respectively, were obtained. For stress RMSEs, the respective values are 55.1±11.6 and 34.4±2.1 MPa.

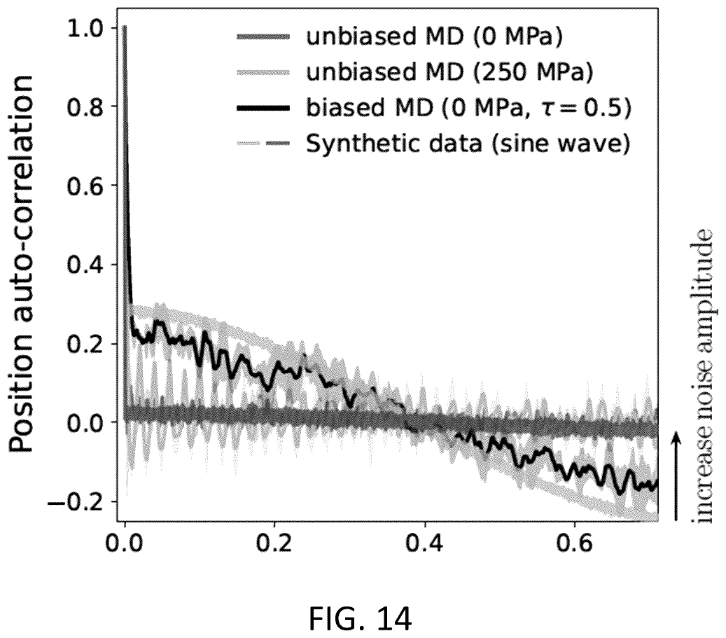

FIG. 14 illustrates that MD simulations driven by bias stress according to embodiments of the present invention often demonstrate correlated moves leading to enhanced exploration of the respective phase space, which is different from conventional approaches, typically showing a random walk-like behavior.

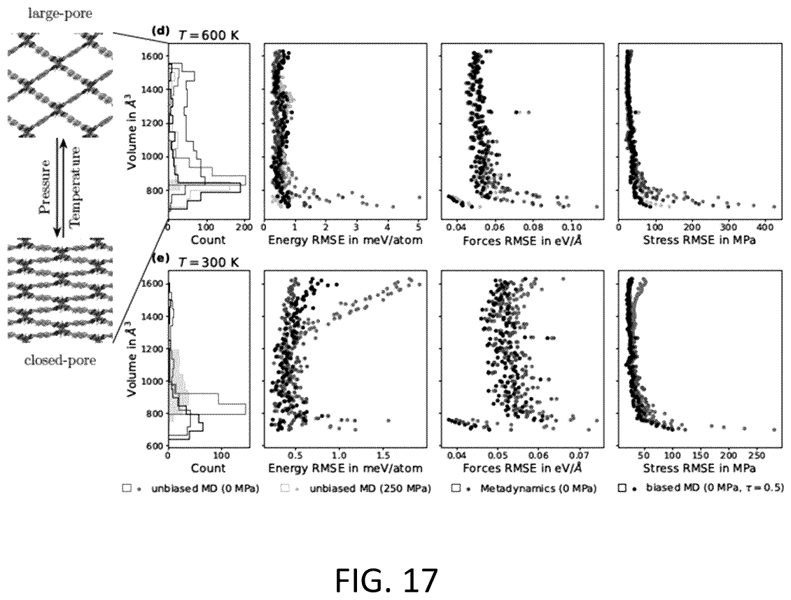

FIG. 17 is a comparison of AL approaches employing biased and unbiased MD simulations to generate the candidate pool of atomic configurations, illustrating that uncertainty-biased MD simulations according to embodiments of the present invention advantageously cover larger configurational space compared to their counterparts and achieve uniform distribution of energy, atomic force, and stress RMSE.

Embodiments of the present invention provide a novel AL approach to the data-driven development of informative yet minimal data sets for training MLIPs. Ensemble-free uncertainties based on sketched gradient features are employed to drive MD simulations towards high-uncertainty regions—a technique to enhance AL pipelines. Ensemble-free atom-based uncertainties are introduced, derived from sketched gradient features, and employ CP for their calibration. Combining (total) ensemble-free uncertainties with AD, a more general way to perform uncertainty-driven MD simulations can be proposed, and a technique for exploring pressure-induced phase transitions in material systems can be introduced.

The extent to which uncertainty-driven simulations enhance the generation of atomic data sets is investigated, aiming to develop MLIPs that exhibit uniform accuracy across the entire phase space. This objective is closely related with rare event sampling, differing in its emphasis on uniform error distribution rather than on uniform phase space coverage. The introduced UDAL approach can be assessed using two benchmark systems: alanine dipeptide and MIL-53(Al). The former is a molecular system frequently used to assess the performance of enhanced sampling techniques. The latter is a flexible meta organic framework, that features closed- and large-pore stable phases.

The performance of introduced atom-based ensemble-free uncertainties is evaluated in data-scare and data-rich regimes using both benchmark systems, alanine dipeptide and MIL-53(Al). The uncertainties of embodiments of the present invention were observed to correlate well with RMSEs of atomic forces in both regimes, with correlation coefficients on par with or better than those obtained with the uncertainty of an ensemble of three models. The performance of ensemble-based uncertainty could be improved by increasing the number of ensemble members. However, this improvement may come with increased computational cost, unfavorable for the AL setting, where an unlabeled pool of data is generated dynamically. Calibrating the uncertainties by employing CP technique helps align the highest error in atomic forces with an equivalent uncertainty value. This property is crucial in some settings for preventing, e.g., an MD simulation entering highly extrapolative regions and triggering first-principle calculation solely on meaningful atomic configurations.

Applying UDAL approaches to alanine dipeptide, the data-driven exploration of its CVs, φ and ψ, were investigated. Uncertainty-driven MD, run at 300 K, were observed to explore effectively the entire space of CVs at a rate comparable to high-temperature simulations, performed at 1200 K. This result serves as a first evidence of uncertainty-driven MD enabling the exploration of larger portions of the configurational space of complex systems in a given amount of simulation time. Uncertainty-driven exploration of phase spaces is a data driven approach which ensures the continuous decrease of the error level across the entire phase space. Thus, it converges to a uniformly accurate MLIPs, in contrast to, e.g., metadynamics which leads to uniform sampling the space of CVs and a diffusive motion along the latter at convergence.

The improvement in energies and atomic forces RMSEs correlates with the exploration of the CV space. Similarly performing models were obtained when using uncertainty-driven and high-temperature AL, outperforming those trained on data sets generated at 300 and 600 K by a factor of more than 13 and 1.6 in energies and atomic forces, respectively. The provided results clearly show the advantage of UDAL over its conventional counterparts. They achieved the desired performance without any prior knowledge on the system, simplifying the application of MLIPs to complex and yet unexplored molecular systems. Compared to an ensemble of three models, achieving the performance of the latter could be achieved at a fraction of its computational cost. A primary advantage of employing an ensemble at inference remains the heuristic measure of stability during MD, based mainly on defining bond distance ranges in a systems. This feature of an ensemble leads to improved rates of the exploration of CV spaces at early AL iterations.

Using AD, a novel biasing technique was derived for investigating pressure-induced phase transitions in material systems. The proposed approach utilizes the bias stress for reducing the internal pressure of a system during an MD simulation in the NpT statistical ensemble, facilitating the exploration of phase transitions. The proposed technique was applied to enhance exploration of the phase space of MIL-53(Al) and developed an uniformly accurate interatomic potential for it. The UDAL embodiment of the present invention was observed to lead to more accurate inter-atomic potentials compared to those trained on data generated using incremental learning combined with metadynamics simulations. This result may be attributed to the fact that metadynamics imposes uniform sampling of cell parameters, while uncertainty-driving leads to uniform error distribution across the entire phase space.

The investigated biasing technique proves to enable phase transition between two stable states of MIL-53(Al), as shown by experiments initialized from models pre-trained on closed-pore configurations. While naive zero-pressure dynamics could not explore the large-pore state, embodiments of the present invention lead to an effective exploration of the latter, on par with high-pressure simulations. Comparing the ensemble-free approaches of embodiments of the present invention with the results obtained for an ensemble of three models, the former outperform the latter in energy, atomic forces, and stress RMSEs, while being more than two times more computationally efficient. For stress RMSEs, an improvement of a factor of 1.7 was found, using a posterior-based approach. Uncertainty-driven approaches, being very efficient in exploring phase spaces and developing uniformly accurate MLIPs, come with an additional computational cost, compared to naive AL. At inference they require computational times larger by a factor of 1.3-1.7 compared to the latter, attributed to additional gradient calculation.

Even though embodiments provide a comprehensive investigation of UDAL approaches, further embodiments may improve the extent to which batch selection algorithms improve the performance of final models, providing a maximally diverse training data set. Furthermore, further embodiments including developed training data sets may lead to improved stability during MD simulations, going beyond some heuristic measures. Finally, further embodiments including biasing dynamics with atom-based uncertainties might enhance the exploration of local environments, employed for modeling interatomic interactions by most cutting-edge MLIPs.

Embodiments of the present invention were investigated for a new paradigm for data set generation, facilitating the development of high-quality interatomic potential models for chemically complex atomic systems. An uncertainty-driven active learning approach is provided which goes beyond traditional enhanced sampling techniques and naive active learning with ensembles of inter-atomic potentials for uncertainty quantification. The approaches of embodiments of the present invention hold the potential to broaden the applications of machine learning for investigating material systems, both those of current interest and those yet to be explored.

Gaussian moment neural network: the Gaussian moment neural network (GM-NN) approach employs an artificial NN to map a local atomic environment Sᵢ to the atomic energy Eᵢ(Sᵢ, θ). GM-NN uses a fully-connected feed-forward NN with two hidden layers:

$${y}_{i} = 0.1 \cdot {b}^{( 3 )} + \frac{1}{\sqrt{{d}_{2}}} {W}^{( 3 )} ϕ ( 0.1 \cdot {b}^{( 2 )} + \frac{1}{\sqrt{{d}_{1}}} {W}^{2} ϕ ( 0.1 \cdot {b}^{( 1 )} + \frac{1}{\sqrt{{d}_{0}}} {W}^{( 1 )} {G}_{i} ) ) \qquad ( 10 )$$

with W^{(l+1)}∈ [symbol] ^{d}^{ₗ₊₁}^{×d}^{ₗ} and b^{(l+1)}∈ [symbol] ^{d}^{ₗ₊₁} representing the weights and biases of layer l+1. Embodiments can employ a NN with d₀=910 input neurons (corresponding to the dimension of the input feature vector Gᵢ=Gᵢ (Sᵢ), d₁=d₂=512 hidden neurons, and a single output neuron, d₃=1. The network's weights W^{(l+1)} can be initialized by selecting entries from a normal distribution with zero mean and unit variance. The trainable bias vectors b^{(l+1)} are initialized to zero. To improve the accuracy and convergence of the GM-NN model, a neural tangent parameterization (factors of 0.1 and 1/\sqrt{dₗ}) can be implemented. For the activation function φ, the Swish/SiLU function can be used. To aid the training process, the output of the NN can be scaled and shifted, Eᵢ(Sᵢ, θ)=c·(ρ_{Z}_{ᵢ}yᵢ+μ_{Z}_{ᵢ}) (11), where the trainable shift parameters μ_{Z}_{ᵢ} are initialized by solving a linear regression problem, and the trainable scale parameters ρ_{Z}_{ᵢ} are initialized to 1. The constant c is determined by the RMS error per atom of the regression solution.

GM-NN models can employ a Gaussian moment (GM) representation to encode the invariance of total energy with respect translations, rotations, and permutations of the same species. By computing pairwise distance vectors rᵢⱼ=rᵢ−rⱼ and then splitting them into radial and angular components, denoted as rᵢⱼ=∥rᵢⱼ∥2 and [symbol] =rᵢⱼ/rᵢⱼ, respectively, the GMs can be obtained as follows:

$${Ψ}_{i , L , s} = {\sum }_{j \neq i} {R}_{{Z}_{i , {Z}_{j} , s}} ( {r}_{ij} , β ) \text{?} , \qquad ( 12 )$$

$$where {\hat{r}}_{ij}^{⊗ L} = \text{?} ⊗ … ⊗ {\hat{r}}_{ij}^{⊗ L}$$

is the L-fold outer product. The nonlinear radial functions R_{Z}_{ᵢ}_{,Z}_{ⱼ}_{,s} (rᵢⱼ, β) are defined as a sum of Gaussian functions Φ_{s′}(rᵢⱼ) (N_{Gauss}=9),

$${R}_{{Z}_{i , {Z}_{j} , s}} ( {r}_{ij} , β ) = \frac{1}{\sqrt{{N}_{G a u s s}}} {\sum }_{{s}^{′} = 1}^{{N}_{G a u s s}} {β}_{{Z}_{i , {Z}_{j} , s , {s}^{′}}} {Φ}_{{s}^{′}} ( {r}_{i j} ) . \qquad ( 13 )$$

The factor 1/\sqrt{N_{Gauss}} impacts the effective learning rate, inspired by neural tangent parametrization. The radial functions are centered at equidistantly spaced grid points ranging from Rₘᵢₙ=0.5 Å to R_{cut}, set to 5.0 and 6.0 Å for alanine dipeptide and MIL-53(Al), respectively. The radial functions are re-scaled by a cosine cutoff function to ensure a smooth dependence on the number of atoms within the cutoff sphere. Chemical information is embedded in the GM representation through trainable parameters β_{Z}_{ᵢ}_{,Z}_{ⱼ}_{,s,s′}, with the index s iterating over the number of independent radial basis functions (N_{basis}=7).

Features invariant to rotations, Gi, are obtained by computing full tensor contractions of tensors defined by Eq. (12), e.g.,

$${G}_{i , {s}_{1} , {s}_{2} , {s}_{3}} = {( {Ψ}_{i , 1 , {s}_{1}} )}_{a} {( {Ψ}_{i , 1 , {s}_{2}} )}_{b} {( {Ψ}_{i , 2 , {s}_{3}} )}_{a , b} , \qquad ( 14 )$$

where Einstein notation is used, i.e., the right-hand sides are summed over a,b∈{1,2,3}. Specific full tensor contractions can be defined by using generating graphs. In a practical implementation, all GMs can be computed at once and reduce the number of invariant features based on the permutational symmetries of the respective graphs.

All parameters θ={W, b, β, ρ, μ} of the NN are optimized by minimizing the combined squared loss on training data [symbol] ₜᵣₐᵢₙ=(Xₜᵣₐᵢₙ, Υₜᵣₐᵢₙ), with

$${X}_{t r a i n} = {\{ {S}^{( k )} \}}_{k = 1}^{{N}_{t r a i n}} and {Υ}_{train} = {\{ {E}_{k}^{r e f} , {\{ {F}_{i , k}^{r e f} \}}_{i = 1}^{{N}_{a t}} , {σ}_{k}^{r e f} \}}_{k = 1}^{{N}_{t r a i n}} , \qquad ( 15 )$$

$$ℒ ( θ ; {𝒟}_{t r a i n} ) = {\sum }_{k = 1}^{{N}_{train}} [ {C}_{e} {‖ {E}_{k}^{r e f} - E ( {S}^{( k )} , θ ‖}_{2}^{2} + \frac{{C}_{f}}{3 {N}_{a t}^{( k )}} {\sum }_{i = 1}^{{N}_{a t}^{( k )}} {‖ {F}_{i ,}^{ref} - {F}_{i} ( {S}^{( k )} , θ ) ‖}_{2}^{2} + \frac{{C}_{s} {V}_{k}}{6} {‖ {σ}_{k}^{ref} - σ ( {s}^{( k )} , θ ) ‖}_{2}^{2} ] .$$

Cₑ=1.0, C_{f}=4.0 Å², and Cₛ=0.01 have been chosen to balance the relative contributions of energies, forces, and stresses, respectively.

Using AD, atomic forces can be computed as negative gradients of total energy with respect to atomic coordinates: Fᵢ(S^{(k)}, θ)=−∇ᵣ_{ᵢ}E(S^{(k)}, θ). (16)

Furthermore, AD can be used to compute stress tensor, defined by:

$$σ ( {S}^{( k )} , θ ) = \frac{1}{{V}_{k}} {∇}_{ε} E ( {S}^{( k )} , θ , ε ) {\vert}_{ε = 0} , \qquad ( 17 )$$

where E (S^{(k)}, θ, ε), is total energy after a strain deformation with symmetric tensor ε∈ [symbol] ^{3×3}, i.e., r→(1+)·r. As stress tensor is symmetric, only its upper triangular part is used in the loss function. Here, Vₖ is the volume of the periodic cell.

The Adam optimizer (see Diederik P. Kingma, Jimmy Ba. Adam: A Method for Stochastic Optimization. arXiv:1412.6980 (2014)) can be employed to minimize the loss function. The respective parameters of the optimizer are β₁=0.9, β₂=0.999, and ε=10⁻⁷. Usually, a mini batch of 32 molecules is used. Yet, in the initial active learning iterations, smaller mini-batches were used because the training data sizes were less than 32. The layer-wise learning rates are set to 0.03 for the parameters of the fully connected layers, 0.02 for the trainable representation, as well as 0.05 and 0.001 for the shift and scale parameters of atomic energies, respectively. The training is performed for 1000 training epochs. To prevent overfitting during training, the early stopping technique was employed. All models were trained within the PyTorch framework.

Sketched gradient features: To derive atomic gradient features, gradients of Eq. (1) are computed with respect to the parameters of the fully connected layers in Eq. (10). In the following, the product structure of atomic gradient features is employed. However, to obtain the latter, the network in Eq. (10) can be rewritten as follows:

$${z}_{i}^{( l + 1 )} = {\hat{W}}^{( l + 1 )} {\hat{x}}_{i}^{( l )} ∈ {ℝ}^{{d}_{l} + 1} , \qquad ( 18 )$$

$${\hat{W}}^{( l + 1 )} = ( {W}^{( l + 1 )} , {b}^{( l + 1 )} ) ∈ {ℝ}^{{d}_{l} + 1 \times ( {d}_{l} + 1 )} ,$$

$${\hat{x}}_{i}^{( l )} = {( \frac{1}{\sqrt{{d}_{l}}} {x}_{i}^{( l )} , 0.1 )}^{T} ∈ {ℝ}^{{d}_{l} + 1} ,$$

where z^{(l)} and x^{(l)} are the pre- and post-activation vectors of layer l. Thus, atomic gradient features φ(Sᵢ) read:

$$ϕ ( {S}_{i} ) = ( \frac{\partial {z}_{i}^{( L )}}{\partial {W}^{( 1 )}} , … \frac{\partial {Z}_{i}^{( L )}}{\partial {\hat{W}}^{( L )}} ) = ( \frac{\partial {Z}_{i}^{( L )}}{\partial {Z}_{i}^{( 1 )}} ⊗ {\tilde{x}}_{i}^{( 0 )} , … , \frac{\partial {z}_{i}^{( L )}}{\partial {z}_{i}^{( L )}} ⊗ {\tilde{x}}_{i}^{( L - 1 )} ) . \qquad ( 19 )$$

To make the calculation of the latter computationally tractable, a random projections (sketching) is employed. For atomic gradient features φ(Sᵢ)∈R^{d}^{_{feat}} and a random matrix U∈ [symbol] ^{d}^{ᵣₚ}^{×d}^{_{feat}}—with d_{feat} and dᵣₚ representing the number of atomic features and random projections, respectively—randomly projected atomic gradient features can be defined as:

$$ϕ ( {S}_{i} ) \to U ϕ ( {S}_{i} ) ∈ {ℝ}^{d r p} , \qquad ( 20 ) .$$

Here, the elements of U are drawn from a normal distribution with zero mean and unit variance. Embodiments can use dᵣₚ=512, where:

$${( {ϕ}_{1} , {ϕ}_{2} )}^{T} \to {U}_{1} {ϕ}_{1} + {U}_{2} {ϕ}_{2} , {ϕ}_{1} ⊗ {ϕ}_{2} \to ( {U}_{1} {ϕ}_{1} ) ⊙ ( {U}_{2} {ϕ}_{2} ) , \qquad ( 21 )$$

with ⊙ denoting the element-wise or Hadamard product. Using the above expressions, sketched atomic gradient features were obtained of

$$ϕ ( {S}_{i} ) \to {\sum }_{l = 1}^{L} ( {U}_{o u t}^{( l )} {ϕ}_{o u t}^{( l )} ( {S}_{i} ) ) ⊙ ( {U}_{i n}^{( l - 1 )} {ϕ}_{i n}^{( l - 1 )} ( {S}_{i} ) ) , \qquad ( 22 ) ,$$

with

$${ϕ}_{o u t}^{( l )} ( {S}_{i} ) = \frac{\partial {Z}_{i}^{( l )}}{\partial {z}_{i}^{( l )}} and {ϕ}_{i n}^{( l )} ( {S}_{i} ) = {\tilde{x}}_{i}^{( l )} .$$

### All

$${U}_{i n}^{( l )} and {U}_{o u t}^{( l )}$$

are sampled independently.

For atom-based uncertainties the sketched atomic gradient features can be used directly. For (total) uncertainties, a sum

$$ϕ ( S ) = {\sum }_{i = 1}^{{N}_{a t}} ϕ$$

(Sᵢ) is worked with. Thus, embodiments can use that the individual projections (rows of Eq. (22)) are linear in the features and obtain for the (total) gradient features:

$$ϕ ( S ) = {\sum }_{i = 1}^{{N}_{a t}} {\sum }_{l = 1}^{L} ( {U}_{o u t}^{( l )} {ϕ}_{o u t}^{( l )} ( {S}_{i} ) ) ⊙ ( {U}_{i n}^{( l - 1 )} {ϕ}_{i n}^{( l - 1 )} ( {S}_{i} ) ) , \qquad ( 23 ) ,$$

given that all of the individual random projections use the same random matrices.

Selection methods: The simplest batch selection method is based on querying points only by their uncertainty values. Specifically, given the already selected structures X_{batch} for an unlabeled pool Xₚₒₒₗ, the next point can be selected by:

$$S = {arg max}_{S ∈ {X}_{p ool} ∖ {X}_{b a t c h}} u ( S ) , \qquad ( 24 )$$

until N_{batch} structures are selected. Embodiments can use this selection method combined with the ensemble-based uncertainty.

For the posterior-based uncertainty, embodiments can constrain diversity of the selected batch by using the covariance between structures, as follows:

$$Cov ( S , {S}^{′} ; {𝒟}_{t r a i n} ) = {λ}^{2} {ϕ ( S )}^{T} {( {ϕ ( {X}_{t r a i n} )}^{T} ϕ ( {X}_{train} ) + {λ}^{2} I )}^{- 1} ϕ ( {S}^{′} ) \qquad ( 25 )$$

The corresponding method selects structures in a greedy fashion, i.e., one structure per iteration, such that the determinant of the covariance matrix is maximized:

$$S = {arg max}_{S ∈ {X}_{p ool} ∖ {X}_{b a t c h}} det [ Cov ( {𝒳}_{b a t c h} ⋃ \{ S \} , {𝒳}_{b a t c h} ⋃ \{ S \} ; {𝒟}_{t r a i n} ) ] \qquad ( 26 )$$

Embodiments can combine this selection method with posterior-based uncertainty. For the distance-based uncertainty, the diversity of the acquired batch was ensured by greedily selecting structures with a maximum distance to all previously selected points and training points. The respective selection method reads:

$$S = {arg max}_{S ∈ {X}_{p ool} ∖ {X}_{b a t c h}} {min}_{{S}^{′} ∈ {X}_{train} ⋃ {X}_{b a t c h}} {‖ ϕ ( S ) - ϕ ( {S}^{′} ) ‖}_{2}^{2} \qquad ( 27 )$$

Embodiments can combine this selection method with distance-based uncertainty. Embodiments can also apply this batch selection method to define the most representative subset of atomic features when calculating atom-based uncertainty derived from feature space distances.

Conformal prediction: In the following, the statement concerning the alignment between the largest prediction error and the uncertainty of the corresponding prediction is formalized. Technically, this property can be referred to as finite sample coverage, defined as:

$$ℙ \{ {y}_{t e s t} ∈ C ( {x}_{t e s t} ) \} \geq 1 - α . \qquad ( 28 )$$

Here, (xₜₑₛₜ,yₜₑₛₜ) are the newly observed data, while C defines the prediction set based on previous observations

$${\{ ( {x}_{k} , {y}_{k} ) \}}_{k = 1}^{{N}_{c a l i b r}} .$$

The hyper-parameter α is the user-chosen confidence level. CP guarantees that the prediction set contains the true label with a probability of almost 1−α.

Density functional theory calculations: All DFT calculations were performed using CP2K simulation package (version 2023.1). To maintain coherence with reference incremental learning calculations, the PBE functional was employed with Grimme D3 dispersion correction. A hybrid basis set, combining TZVP Gaussian basis functions and plane waves, was employed. GTH pseudopotentials were used to smoothen the electron density near the nuclei. To ensure the convergence of force and stress calculations, a plane wave cutoff energy of 1000 Ry was selected.

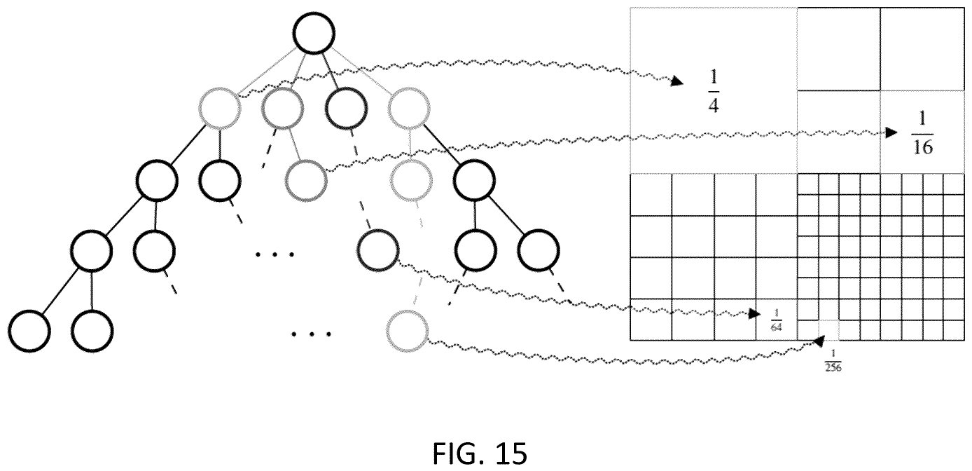

Coverage of collective variable space: To measure how well different methods explore the (bounded) space of interest, a tree-based weighted recursive partitioning of a d-dimensional Euclidean space was implemented, which is reminiscent of quadtrees and matrix-based octrees, but allows to choose how many times n to split each dimension. Thus the variety of the tree is k=n^{d}. Each node of this complete, k-ary tree encodes a generalized hypercube of d-dimensions, where each side length depends on the boundaries of the original space. The root node represents the full bounded space. A tree of height L has total number of partitions equal to (k^{L+1}−1)/(k−1), and each level [symbol] has [symbol] nodes. The hyper-parameters chosen are n=2, d=2 (for the CVs φ and ψ of alanine dipeptide) and L=5, for a total of 1365 partitions of the space of interest. The proposed surface coverage metric of embodiments of the present invention can use this data structure as a proxy to capture how many partitions of the space a method can explore in the least amount of iterations. At the same time, methods that get stuck in a region of the space exploring partitions of smaller volumes should be penalized, that is, those represented by nodes at deeper levels in the tree. For this reason, each node at level l is associated a reward (or weight) of 1/ [symbol] , so that each level of the tree has a cumulative reward of 1. It follows that the optimal strategy could be to perform a breadth-first search of the nodes of this tree, which translates into observing the largest partitions of unobserved space first. In addition, partitions that are visited again by the methods give no additional reward, so there is no gain in getting stuck in a certain partition. FIG. 15 visually represents the algorithm in for the simple case of d=2.

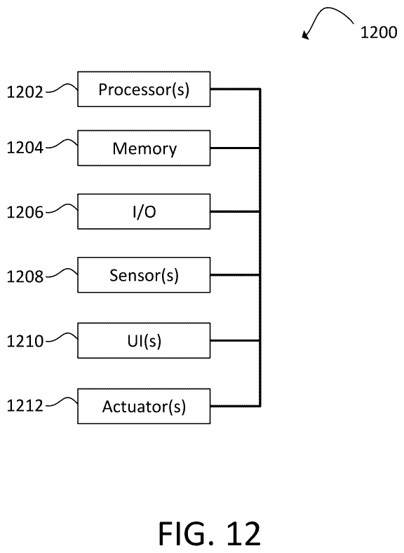

Referring to FIG. 12, a processing system 1200 can include one or more processors 1202, memory 1204, one or more input/output devices 1206, one or more sensors 1208, one or more user interfaces 1210, and one or more actuators 1212. Processing system 1200 can be representative of each computing system disclosed herein.

Processors 1202 can include one or more distinct processors, each having one or more cores. Each of the distinct processors can have the same or different structure. Processors 1202 can include one or more central processing units (CPUs), one or more graphics processing units (GPUs), circuitry (e.g., application specific integrated circuits (ASICs)), digital signal processors (DSPs), and the like. Processors 1202 can be mounted to a common substrate or to multiple different substrates.

Processors 1202 are configured to perform a certain function, method, or operation (e.g., are configured to provide for performance of a function, method, or operation) at least when one of the one or more of the distinct processors is capable of performing operations embodying the function, method, or operation. Processors 1202 can perform operations embodying the function, method, or operation by, for example, executing code (e.g., interpreting scripts) stored on memory 1204 and/or trafficking data through one or more ASICs. Processors 1202, and thus processing system 1200, can be configured to perform, automatically, any and all functions, methods, and operations disclosed herein. Therefore, processing system 1200 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 1200 can be configured to perform task “X”. Processing system 1200 is configured to perform a function, method, or operation at least when processors 1202 are configured to do the same.

Memory 1204 can include volatile memory, non-volatile memory, and any other medium capable of storing data. Each of the volatile memory, non-volatile memory, and any other type of memory can include multiple different memory devices, located at multiple distinct locations and each having a different structure. Memory 1204 can include remotely hosted (e.g., cloud) storage.

Examples of memory 1204 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 1204.

Input-output devices 1206 can include any component for trafficking data such as ports, antennas (i.e., transceivers), printed conductive paths, and the like. Input-output devices 1206 can enable wired communication via USB®, DisplayPort®, HDMI®, Ethernet, and the like. Input-output devices 1206 can enable electronic, optical, magnetic, and holographic, communication with suitable memory 1206. Input-output devices 1206 can enable wireless communication via WiFi®, Bluetooth®, cellular (e.g., LTE®, CDMA®, GSM®, WiMax®, NFC®), GPS, and the like. Input-output devices 1206 can include wired and/or wireless communication pathways.

Sensors 1208 can capture physical measurements of environment and report the same to processors 1202. User interface 1210 can include displays, physical buttons, speakers, microphones, keyboards, and the like. Actuators 1212 can enable processors 1202 to control mechanical forces.

Processing system 1200 can be distributed. For example, some components of processing system 1200 can reside in a remote hosted network service (e.g., a cloud computing environment) while other components of processing system 1200 can reside in a local computing system. Processing system 1200 can have a modular design where certain modules include a plurality of the features/functions shown in FIG. 12. For example, I/O modules can include volatile memory and one or more processors. As another example, individual processor modules can include read-only-memory and/or local caches.

### REFERENCES

Butler, K. T., Davies, D. W., Cartwright, H., Isayev, O. & Walsh, A. Machine learning for molecular and materials science. Nature 559, 547-555, DOI: 10.1038/s41586-018-0337-2 (2018).
Xie, Y. et al. Uncertainty-aware molecular dynamics from bayesian active learning for phase transformations and thermal transport in sic. npj Comput. Mater. 9, 36, DOI: 10.1038/s41524-023-00988-8 (2023).
Gubaev, K. et al. Performance of two complementary machine-learned potentials in modelling chemically complex systems. npj Comput. Mater. 9, 129, DOI: 10.1038/s41524-023-01073-w (2023).
Friederich, P., Häse, F., Proppe, J. & Aspuru-Guzik, A. Machine-learned potentials for next-generation matter simulations. Nat. Mater. 20, 750-761 (2021).
Unke, O. T. et al. Machine Learning Force Fields. Chem. Rev. 121, 10142-10186, DOI: 10.1021/acs. chemrev.0c01111 (2021).
Rupp, M., Tkatchenko, A., Müller, K.-R. & von Lilienfeld, O. A. Fast and Accurate Modeling of Molecular Atomization Energies with Machine Learning. Phys. Rev. Lett. 108, 058301, DOI: 10.1103/PhysRevLett.108.058301 (2012).
Faber, F. A., Christensen, A. S., Huang, B. & von Lilienfeld, O. A. Alchemical and structural distribution based representation for universal quantum machine learning. J. Chem. Phys. 148, 241717, DOI: 10.1063/1.5020710 (2018).
Shapeev, A. V. Moment tensor potentials: A class of systematically improvable interatomic potentials. Multiscale Model. Simul. 14, 1153-1173, DOI: 10.1137/15M1054183 (2016).
Drautz, R. Atomic cluster expansion for accurate and transferable interatomic potentials. Phys. Rev. B 99, 014104 (2019).
Bartók, A. P., Payne, M. C., Kondor, R. & Csányi, G. Gaussian approximation potentials: The accuracy of quantum mechanics, without the electrons. Phys. Rev. Lett. 104, 136403, DOI: 10.1103/PhysRevLett.104.136403 (2010).
Bartók, A. P., Kondor, R. & Csányi, G. On representing chemical environments. Phys. Rev. B 87, 184115, DOI: 10.1103/PhysRevB.87.184115 (2013).
Behler, J. & Parrinello, M. Generalized Neural-Network Representation of High-Dimensional Potential-Energy Surfaces. Phys. Rev. Lett. 98, 146401, DOI: 10.1103/PhysRevLett.98.146401 (2007).
Artrith, N. & Urban, A. An implementation of artificial neural-network potentials for atomistic materials simulations: Performance for TiO2. Comput. Mater. Sci. 114, 135-150, DOI: https://doi.org/10.1016/j.commatsci.2015.11.047 (2016).
Smith, J. S., Isayev, O. & Roitberg, A. E. Ani-1: an extensible neural network potential with dft accuracy at force field computational cost. Chem. Sci. 8, 3192-3203, DOI: 10.1039/C6SC05720A (2017).
Schütt, K. et al. Schnet: A continuous-filter convolutional neural network for modeling quantum interactions. In Guyon, I. et al. (eds.) NeurIPS, vol. 30, 991-1001 (Curran Associates, Inc., 2017).
Zaverkin, V. & Kästner, J. Gaussian Moments as Physically Inspired Molecular Descriptors for Accurate and Scalable Machine Learning Potentials. J. Chem. Theory Comput. 16, 5410-5421, DOI: 10.1021/acs.jctc.0c00347 (2020).
Schütt, K. T., Unke, O. T. & Gastegger, M. Equivariant message passing for the prediction of tensorial properties and molecular spectra. ICML 1-13 (2021).
Zaverkin, V., Holzmüller, D., Steinwart, I. & Kästner, J. Fast and Sample-Efficient Interatomic Neural Network Potentials for Molecules and Materials Based on Gaussian Moments. J. Chem. Theory Comput. 17, 6658-6670, DOI: 10.1021/acs.jctc.1c00527 (2021).
Batzner, S. et al. E (3)-equivariant graph neural networks for data-efficient and accurate interatomic potentials. Nat. Commun. 13, 2453 (2022).
Li, Z., Kermode, J. R. & De Vita, A. Molecular dynamics with on-the-fly machine learning of quantum-mechanical forces. Phys. Rev. Lett. 114, 096405 (2015).
Podryabinkin, E. V. & Shapeev, A. V. Active learning of linearly parametrized interatomic potentials. Comput. Mater. Sci. 140, 171-180, DOI: https://doi.org/10.1016/j.commatsci.2017.08.031 (2017).
Gastegger, M., Behler, J. & Marquetand, P. Machine learning molecular dynamics for the simulation of infrared spectra. Chem. Sci. 8, 6924-6935, DOI: 10.1039/C7SC02267K (2017).
Zhang, L., Lin, D.-Y., Wang, H., Car, R. & E, W. Active learning of uniformly accurate interatomic potentials for materials simulation. Phys. Rev. Mater. 3, 023804, DOI: 10.1103/PhysRevMaterials.3.023804 (2019). 1112
Vandermause, J. et al. On-the-fly active learning of interpretable bayesian force fields for atomistic rare events. npj Comput. Mater. 6 (2020).
Briganti, V. & Lunghi, A. Efficient generation of stable linear machine-learning force fields with uncertainty aware active learning. Mach. Learn. Sci. Technol. 4, 035005, DOI: 10.1088/2632-2153/ace418 (2023).
Laio, A. & Parrinello, M. Escaping free-energy minima. Proc. Natl. Acad. Sci. 99, 12562-12566 (2002).
Barducci, A., Bussi, G. & Parrinello, M. Well-tempered metadynamics: A smoothly converging and tunable free energy method. Phys. Rev. Lett. 100, 020603 (2008).
Yoo, D., Jung, J., Jeong, W. & Han, S. Metadynamics sampling in atomic environment space for collecting training data for machine learning potentials. npj Comput. Mater. 7, 131, DOI: 10.1038/s41524-021-00595-5 (2021).
Yang, M., Bonati, L., Polino, D. & Parrinello, M. Using metadynamics to build neural network potentials for reactive events: the case of urea decomposition in water. Catal. Today 387, 143-149, DOI: https://doi.org/10.1016/j.cattod.2021.03.018 (2022).
Vandenhaute, S., Cools-Ceuppens, M., DeKeyser, S., Verstraelen, T. & Van Speybroeck, V. Machine learning potentials for metal-organic frameworks using an incremental learning approach. npj Comput. Mater. 9, 19, DOI: 10.1038/s41524-023-00969-x (2023).
Schran, C., Brezina, K. & Marsalek, O. Committee neural network potentials control generalization errors and enable active learning. J. Chem. Phys. 153, 104105, DOI: 10.1063/5.0016004 (2020). https://pubs.aip.org/aip/jcp/article-pdf/doi/10.1063/5.0016004/13902889/104105_1_online.pdf.
Kulichenko, M. et al. Uncertainty-driven dynamics for active learning of interatomic potentials. Nat. Comput. Sci. 3, 230-239, DOI: 10.1038/s43588-023-00406-5 (2023).
Van der Oord, C., Sachs, M., Kovács, D. P., Ortner, C. & Csányi, G. Hyperactive Learning (HAL) for Data-Driven Interatomic Potentials. ArXiv abs/2210.04225 (2022). Janet, J. P., Duan, C., Yang, T., Nandy, A. & Kulik, H. J. A quantitative uncertainty metric controls error in neural network-driven chemical discovery. Chem. Sci. 10, 7913-7922, DOI: 10.1039/C9SC02298H (2019).
Zhu, A., Batzner, S., Musaelian, A. & Kozinsky, B. Fast uncertainty estimates in deep learning interatomic potentials. J. Chem. Phys. 158, 164111, DOI: 10.1063/5.0136574 (2023). https://pubs.aip.org/aip/jcp/article-pdf/doi/10.1063/5.0136574/17060517/164111_1_5.0136574.pdf.
Zaverkin, V. & Kästner, J. Exploration of transferable and uniformly accurate neural network interatomic potentials using optimal experimental design. Mach. Learn. Sci. Technol. 2, 035009, DOI: 10.1088/2632-2153/abe294 (2021).
Zaverkin, V., Holzmüller, D., Steinwart, I. & Kästner, J. Exploring chemical and conformational spaces by batch mode deep active learning. Digit. Discov. 1, 605-620, DOI: 10.1039/D2DD00034B (2022).
Holzmüller, D., Zaverkin, V., Kästner, J. & Steinwart, I. A framework and benchmark for deep batch active learning for regression. J. Mach. Learn. Res. 24, 1-81 (2023).
Lei, J., G'Sell, M., Rinaldo, A., Tibshirani, R. J. & Wasserman, L. Distribution-free predictive inference for regression. ArXiv abs/1604.04173 (2017).
Romano, Y., Patterson, E. & Candès, E. J. Conformalized quantile regression. ArXiv abs/1905.03222 (2019).
Angelopoulos, A. N. & Bates, S. A gentle introduction to conformal prediction and distribution-free uncertainty quantification. ArXiv abs/2107.07511 (2022).
Hu, Y., Musielewicz, J., Ulissi, Z. W. & Medford, A. J. Robust and scalable uncertainty estimation with conformal prediction for machine-learned interatomic potentials. Mach. Learn. Sci. Technol. 3, 045028, DOI: 1187 10.1088/2632-2153/aca7b1 (2022).
Schwalbe-Koda, D., Tan, A. R. & Gómez-Bombarelli, R. Differentiable sampling of molecular geometries with uncertainty-based adversarial attacks. Nat. Commun. 12, 5104, DOI: 10.1038/s41467-021-25342-8 (2021).
Imbalzano, G. et al. Uncertainty estimation for molecular dynamics and sampling. J. Chem. Phys. 154, 74102, DOI: 10.1063/5.0036522 (2021).
Knuth, F., Carbogno, C., Atalla, V., Blum, V. & Scheffler, M. All-electron formalism for total energy strain derivatives and stress tensor components for numeric atom-centered orbitals. Comput. Phys. Comm. 190, 33-50, DOI: https://doi.org/10.1016/j.cpc.2015.01.003 (2015).
Duchi, J., Hazan, E. & Singer, Y. Adaptive subgradient methods for online learning and stochastic optimization. J. Mach. Learn. Res. 12, 2121-2159 (2011).
Kingma, D. P. & Ba, J. Adam: A Method for Stochastic Optimization. ICLR (2015).
Tian, C. et al. ff19sb: Amino-acid-specific protein back-bone parameters trained against quantum mechanics energy surfaces in solution. J. Chem. Theory Comput. 16, 528-552, DOI: 10.1021/acs.jctc.9b00591 (2020). PMID: 31714766, https://doi.org/10.1021/acs.jctc.9b00591.
Doerr, S. et al. Torchmd: A deep learning framework for molecular simulations. J. Chem. Theory Comput. 17, 2355-2363, DOI: 10.1021/acs.jctc.0c01343 (2021). PMID: 33729795, https://doi.org/10.1021/acs.jctc.0c01343. 1214
Larsen, A. H. et al. The atomic simulation environment—a python library for working with atoms. J. Phys. Condens. Matter 29, 273002 (2017).
Perdew, J. P., Burke, K. & Ernzerhof, M. Generalized gradient approximation made simple. Phys. Rev. Lett. 77, 3865-3868, DOI: 10.1103/PhysRevLett.77.3865 (1996).
Grimme, S., Antony, J., Ehrlich, S. & Krieg, H. A consistent and accurate ab initio parametrization of density functional dispersion correction (dft-d) for the 94 elements h-pu. J. Chem. Phys. 132, 154104 (2010).
Melchionna, S., Ciccotti, G. & Holian, B. L. Hoover NPT dynamics for systems varying in shape and size. Mol. Phys. 78, 533-544, DOI: 10.1080/00268979300100371 (1993).
Melchionna, S. Constrained systems and statistical distribution. Phys. Rev. E 61, 6165-6170, DOI: 10.1103/PhysRevE.61.6165 (2000).
Jacot, A., Gabriel, F. & Hongler, C. Neural Tangent Kernel: Convergence and Generalization in Neural Networks. In Bengio, S. et al. (eds.) NeurIPS, vol. 31, 8580-8589 (Curran Associates, Inc., 2018).
Elfwing, S., Uchibe, E. & Doya, K. Sigmoid-Weighted Linear Units for Neural Network Function Approximation in Reinforcement Learning. Neural Netw. 107, 3-11 (2018).
Ramachandran, P., Zoph, B. & Le, Q. V. Searching for activation functions. ICLR (2018).
Suk, T. & Flusser, J. Tensor method for constructing 3d moment invariants. In Real, P., Diaz-Pernil, D., Molina-Abril, H., Berciano, A. & Kropatsch, W. (eds.) Computer Analysis of Images and Patterns, vol. 6855 of Lecture Notes in Computer Science, 212-219 (Springer, 2011).
Prechelt, L. Early stopping—but when? In Montavon, G., Orr, G. B. & Müller, K.-R. (eds.) Neural Networks: Tricks of the Trade: Second Edition, 53-67, DOI: 10.1007/978-3-642-35289-8_5 (Springer, Berlin, Heidelberg, 2012).
Paszke, A. et al. Pytorch: An imperative style, high performance deep learning library. In Wallach, H. et al. (eds.) NeurIPS, vol. 32, 8024-8035 (Curran Associates, Inc., 2019).
Woodruff, D. P. Sketching as a tool for numerical linear algebra. ArXiv abs/1411.4357, 1-139 (2014).
Kirsch, A., Van Amersfoort, J. & Gal, Y. Batchbald: Efficient and diverse batch acquisition for deep bayesian active learning. NeurIPS 32, 7026-7037 (2019).
Sener, O. & Savarese, S. Active learning for convolutional neural networks: A core-set approach. ICLR (2018).
Kühne, T. D. et al. CP2K: An electronic structure and molecular dynamics software package—Quickstep: Efficient and accurate electronic structure calculations. J. Chem. Phys. 152, 194103, DOI: 10.1063/5.0007045 (2020). https://pubs.aip.org/aip/jcp/article-pdf/doi/10.1063/5.0007045/16718133/194103_1_online.pdf.
Lippert, G., Hutter, J. & Parrinello, M. A hybrid Gaussian and plane wave density functional scheme. Mol. Phys. 92, 477-487 (1997).
Goedecker, S., Teter, M. & Hutter, J. Separable dual space gaussian pseudopotentials. Phys. Rev. B 54, 1703-1710, DOI: 10.1103/PhysRevB.54.1703 (1996).
Finkel, R. A. & Bentley, J. L. Quad trees a data structure for retrieval on composite keys. Acta Inform. 4, 1-9 (1974).
Meagher, D. J. Octree encoding: A new technique for the representation, manipulation and display of arbitrary 3-d objects by computer (Electrical and Systems Engineering Department Rensselaer Polytechnic, 1980).

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications can be made, by those of ordinary skill in the art, within the scope of the following claims, which can include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented, machine learning method for training interatomic potentials of an atomic system, the method comprising performing uncertainty-driven active learning by:
running an atomistic simulation of the atomic system using ensemble-free uncertainties to generate unlabeled atomic data, the ensemble-free uncertainties being derived from gradient features and used to terminate the atomistic simulation and bias the atomistic simulation toward unexplored regions of a configurational and/or chemical space of the atomic system; and
performing active learning to select one or more atomic configurations from the unlabeled atomic data using a trajectory of the atomistic simulation and to train a machine learning model for the interatomic potentials using the one or more atomic configurations.

2. The method according to claim 1, wherein the ensemble-free uncertainties include atom-based uncertainties calibrated using conformal prediction with root-mean-squared errors (RMSEs) of atomic forces of the atomic system.

3. The method according to claim 2, wherein the atomistic simulation updates the atomic forces based on the atom-based uncertainties, computes bias forces and updates atomic positions of the atomic system until a stopping criterion is met, at which point the active learning is performed.

4. The method according to claim 3, wherein the stopping criterion is reached when the atomic forces of the atomic system with updated atomic positions are predicted to have larger RMSEs than a previous iteration.

5. The method according to claim 1, wherein the trained machine learning model is used to run a further atomistic simulation for property prediction which does not use uncertainty bias, the property prediction being for an atomic position, an atomic number, a cell parameter or a composition of the atomic system.

6. The method according to claim 5, further comprising computing gradients of the property prediction using automatic differentiation to update the atomic system, and repeating the uncertainty-driven active learning using the updated atomic system as input.

7. The method according to claim 1, further comprising running a plurality of further atomistic simulations in parallel with the atomistic simulation, wherein the one or more atomic configurations are selected using a batch selection mechanism based on concatenated trajectories of the atomistic simulations.

8. The method according to claim 7, wherein the batch selection mechanism uses informativeness, diversity and representativeness as criteria to select the one or more atomic configurations.

9. The method according to claim 1, wherein alchemical gradients of the ensemble-free uncertainties are computed with respect to atomic numbers and used drive the atomistic simulation to unexplored regions of the chemical space of the atomic system.

10. The method according to claim 1, wherein gradients of the ensemble-free uncertainties are computed with respect to atomic positions, strain deformation and/or cell parameters and are used to derive bias forces and/or to change an internal pressure of the atomic system and drive the atomistic simulation to unexplored regions of the configurational space of the atomic system.

11. The method according to claim 1, wherein the ensemble-free uncertainties are computed from gradients computed for each atom of the atomic system and a total gradient, which is a sum of the gradients computed for each atom of the atomic system.

12. The method according to claim 11, wherein the ensemble-free uncertainties include a distance-based uncertainty and a posterior-based uncertainty computed from the gradients, the distance-based uncertainty being computed as a minimum distance from a respective structure to other structures in a space defined by the gradient features, and the posterior-based uncertainty being computed as Gaussian process regression variance using the gradient features.

13. The method according to claim 1, wherein the gradient features are computed using outputs of a multilayer perceptron, the gradient features possessing a product structure, and wherein a dimensionality of the gradient features is reduced using tensor sketching and/or by using last-layer gradient features.

14. A computer system for training interatomic potentials of an atomic system, the computer system comprising one or more hardware processors, which alone or in combination, are configured to provide for execution of uncertainty-driven active learning comprising the following steps:
running an atomistic simulation of the atomic system using ensemble-free uncertainties to generate unlabeled atomic data, the ensemble-free uncertainties being derived from gradient features and used to terminate the atomistic simulation and bias the atomistic simulation toward unexplored regions of a configurational and/or chemical space of the atomic system; and
performing active learning to select one or more atomic configurations from the unlabeled atomic data using a trajectory of the atomistic simulation and to train a machine learning model for the interatomic potentials using the one or more atomic configurations.

15. A tangible, non-transitory computer-readable medium containing instructions for training interatomic potentials of an atomic system, which, upon being executed by one or more processors, provide for execution of uncertainty-driven active learning comprising the following steps:
running an atomistic simulation of the atomic system using ensemble-free uncertainties to generate unlabeled atomic data, the ensemble-free uncertainties being derived from gradient features and used to terminate the atomistic simulation and bias the atomistic simulation toward unexplored regions of a configurational and/or chemical space of the atomic system; and
performing active learning to select one or more atomic configurations from the unlabeled atomic data using a trajectory of the atomistic simulation and to train a machine learning model for the interatomic potentials using the one or more atomic configurations.
